(19) United States
(12) Patent Application Publication
(10) Pub. No.: US 2010/0093756 A1
(43) Pub. Date: Apr. 15, 2010

(54) HETEROARYL SUBSTITUTED THIENO[2,3-d]PYRIMIDINE AND THEIR USE AS ADENOSINE A2a RECEPTOR ANTAGONISTS

(76) Inventors: J. Kent Berbay, Flourtown, PA (US); Devraj Chakravarty, Hillsborough, NJ (US); Kristi Leonard, Flourtown, PA (US); Brian Christopher Shook, Gilbertsville, PA (US); Aihua Wang, Jamison, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/499,370

(22) Filed: Jul. 8, 2009

Related U.S. Application Data
(60) Provisional application No. 61/104,791, filed on Oct. 13, 2008.

(51) Int. Cl. A61K 31/497; C07D 495/02; A61K 31/519; A61P 25/00

(52) U.S. Cl. 514/255.05; 544/278; 514/260.1

(57) ABSTRACT

This invention relates to a novel thieno[2,3-d]pyrimidine, Z, and its therapeutic and prophylactic uses, wherein X, R¹ and R² are defined in the specification. Disorders treated and/or prevented include Parkinson's Disease.

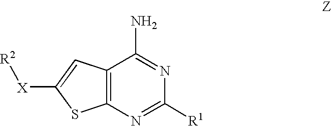

### CROSS-REFERENCE TO RELATED APPLICATIONS

The present application claims the benefits of the filing of U.S. Provisional Application No. 61/104,791 filed Oct. 13, 2008. The complete disclosures of the aforementioned related patent applications are hereby incorporated herein by reference for all purposes.

### FIELD OF THE INVENTION

This invention relates to a novel arylindenopyrimidine and its therapeutic and prophylactic uses. Disorders treated and/or prevented include neurodegenerative and movement disorders ameliorated by antagonizing Adenosine A2a receptors.

### BACKGROUND OF THE INVENTION

Adenosine A2a Receptors Adenosine is a purine nucleotide produced by all metabolically active cells within the body. Adenosine exerts its effects via four subtypes of cell surface receptors (A1, A2a, A2b and A3), which belong to the G protein coupled receptor superfamily (Stiles, G. L. Journal of Biological Chemistry, 1992, 267, 6451). A1 and A3 couple to inhibitory G protein, while A2a and A2b couple to stimulatory G protein. A2a receptors are mainly found in the brain, both in neurons and glial cells (highest level in the striatum and nucleus accumbens, moderate to high level in olfactory tubercle, hypothalamus, and hippocampus etc. regions) (Rosin, D. L.; Robeva, A.; Woodard, R. L.; Guyenet, P. G.; Linden, J. Journal of Comparative Neurology, 1998, 401, 163).

In peripheral tissues, A2a receptors are found in platelets, neutrophils, vascular smooth muscle and endothelium (Gessi, S.; Varani, K.; Merighi, S.; Ongini, E.; Bores, P. A. British Journal of Pharmacology, 2000, 129, 2). The striatum is the main brain region for the regulation of motor activity, particularly through its innervation from dopaminergic neurons originating in the substantial nigra. The striatum is the major target of the dopaminergic neuron degeneration in patients with Parkinson's Disease (PD). Within the striatum, A2a receptors are co-localized with dopamine D2 receptors, suggesting an important site for the integration of adenosine and dopamine signaling in the brain (Fink, J. S.; Weaver, D. Ri; Rivkees, S. A.; Peterfreund, R. A.; Pollack, A. E.; Adler, E. M.; Reppert, S. M. Brain Research Molecular Brain Research, 1992,14,186).

Neurochemical studies have shown that activation of A2a receptors reduces the binding affinity of D2 agonist to their receptors. This D2R and A2aR receptor-receptorinteraction has been demonstrated instriatal membrane preparations of rats (Ferre, S.; con Euler, G.; Johansson, B.; Fredholm, B. B.; Fuxe, K. Proceedings of the National Academy of Sciences I of the United States of America, 1991, 88, 7238) as well as in fibroblast cell lines after transfected with A2aR and D2R cDNAs (Salim, H.; Ferre, S.; Dalal, A.; Peterfreund, R. A.; Fuxe, K.; Vincent, J. D.; Lledo, P. M. Journal of Neurochemistry, 2000, 74, 432). In vivo, pharmacological blockade of A2a receptors using A2a antagonist leads to beneficial effects in dopaminergic neurotoxin MPTP(1-methyl-4-pheny-1,2,3,6-tetrahydropyridine)-induced PC) in various species, including mice, rats, and monkeys (Ikeda, K.; Kurokawa, M.; Aoyana, S.; Kuwana, Y. Journal of Neurochemistry, 2002, 80, 262).

Furthermore, A2a knockout mice with genetic blockade of A2a function have been found to be less sensitive to motor impairment and neurochemical changes when they were exposed to neurotoxin MPTP (Chen, J. F.; Xu, K.; I Petzer, J. P.; Steal, R.; Xu, Y. H.; Beilstein, M.; Sonsalla, P. K.; Castagnoli, K.; Castagnoli, N., Jr.; Schwarsschild, M. A. Journal of Neuroscience, 2001, 1 21, RC 1 43).

In humans, the adenosine receptor antagonist theophylline has been found to produce beneficial effects in PD patients (Mally, J.; Stone, T. W. Journal of the Neurological Sciences, 1995, 132, 129). Consistently, recent epidemiological study has shown that high caffeine consumption makes people less likely to develop PD (Ascherio, A.; Zhang, S. M.; Hernan, M. A.; Kawachi, I.; Colditz, G. A.; Speizer, F. E.; Willett, W. C. Annals of Neurology, 2001, 50, 56). In summary, adenosine A2a receptor blockers may provide a new class of antiparkinsonian agents (Impagnatiello, F.; Bastia, E.; Ongini, E.; Monopoli, A. Emerging Therapeutic Targets, 2000, 4, 635).

Antagonists of the A_{2A} receptor are potentially useful therapies for the treatment of addiction. Major drugs of abuse (opiates, cocaine, ethanol, and the like) either directly or indirectly modulate dopamine signaling in neurons particularly those found in the nucleus accumbens, which contain high levels of A_{2A} adenosine receptors. Dependence has been shown to be augmented by the adenosine signaling pathway, and it has been shown that administration of an A_{2A} receptor antagonist reduces the craving for addictive substances (“The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316) and “Adaptations in Adenosine Signaling in Drug Dependence: Therapeutic Implications”, by Stephen P. Hack and Macdonald J. Christie, Critical Review in Neurobiology, Vol. 15, 235-274 (2003)). See also Alcoholism: Clinical and Experimental Research (2007), 31(8), 1302-1307.

An A_{2A} receptor antagonist could be used to treat attention deficit hyperactivity disorder (ADHD) since caffeine (a non selective adenosine antagonist) can be useful for treating ADHD, and there are many interactions between dopamine and adenosine neurons. Clinical Genetics (2000), 58(1), 31-40 and references therein.

Antagonists of the A_{2A} receptor are potentially useful therapies for the treatment of depression. A_{2A} antagonists are known to induce activity in various models of depression including the forced swim and tail suspension tests. The positive response is mediated by dopaminergic transmission and is caused by a prolongation of escape-directed behavior rather than by a motor stimulant effect. Neurology (2003), 61(suppl 6) S82-S87.

Antagonists of the A_{2A} receptor are potentially useful therapies for the treatment of anxiety. A_{2A} antagonist have been shown to prevent emotional/anxious responses in vivo. Neurobiology of Disease (2007), 28(2) 197-205.

### SUMMARY OF THE INVENTION

Compounds of Formula Z are potent small molecule antagonists of the Adenosine A2a receptor.

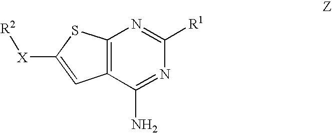

wherein

X is selected from the group consisting of

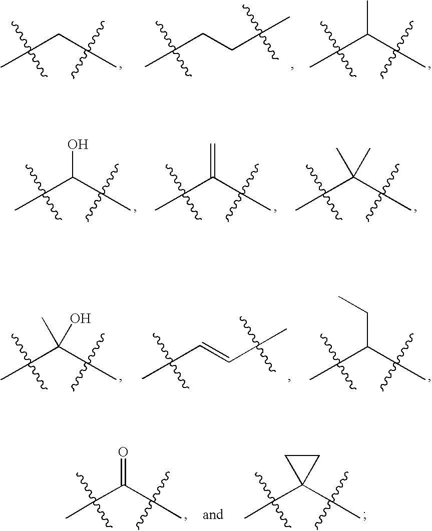

R¹ is heteroaryl which may be substituted with one substituent selected from the group consisting of Cl, Br, F, OH, CN, C₍₁₋₄₎alkyl, CHF₂, CF₃, OCF₃, cyclopropyl, and OC₍₁₋₄₎alkyl;

R² is heteroaryl wherein said heteroaryl is optionally substituted with Cl, F, Br, OC₍₁₋₄₎alkyl, OCF₃, OH, C₍₁₋₄₎alkyl, CHF₂, CF₃, OCH₂CF₃, or a ring selected from the group consisting of:

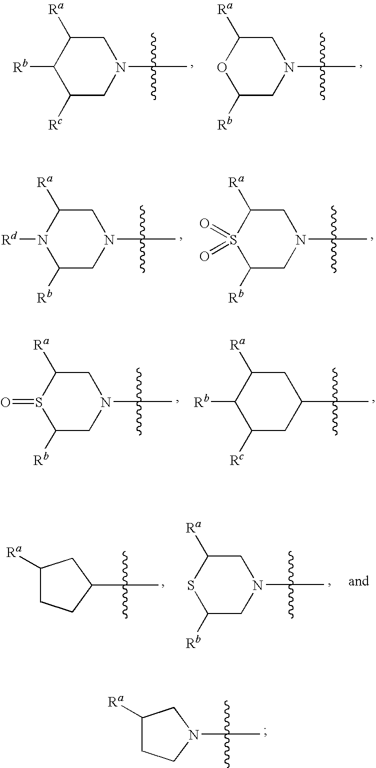

wherein R^{a}, R^{b}, and R^{c} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;

and solvates, hydrates, tautomers and pharmaceutically acceptable salts thereof.

### DETAILED DESCRIPTION OF THE INVENTION

Compounds of Formula Z are potent small molecule antagonists of the Adenosine A2a receptor.

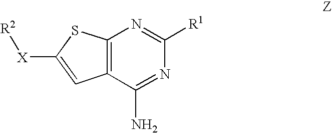

wherein

X is selected from the group consisting of

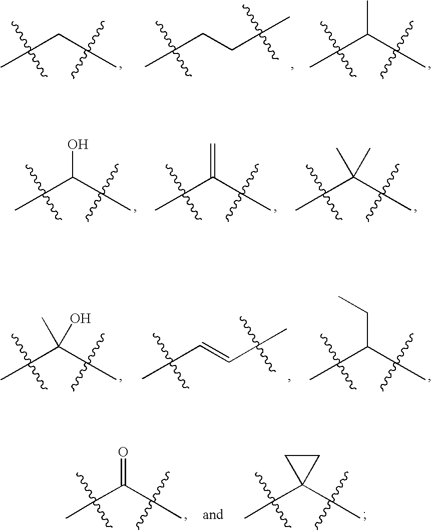

R¹ is heteroaryl which may be substituted with one substituent selected from the group consisting of Cl, Br, F, OH, CN, C₍₁₋₄₎alkyl, CHF₂, CF₃, OCF₃, cyclopropyl, and OC₍₁₋₄₎alkyl;

R² is heteroaryl wherein said heteroaryl is optionally substituted with Cl, F, Br, OC₍₁₋₄₎alkyl, OCF₃, OH, C₁₋₄₎alkyl, CHF₂, CF₃, OCH₂CF₃, or a ring selected from the group consisting of:

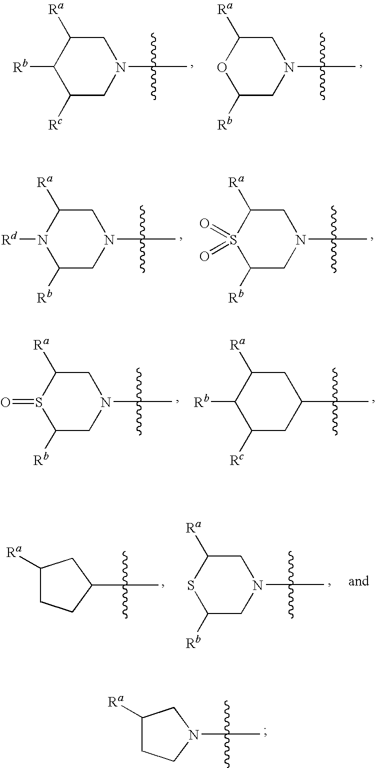

wherein R^{a}, R^{b}, and R^{c} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;

and solvates, hydrates, tautomers and pharmaceutically acceptable salts thereof.

In another embodiment of the invention:

X is selected from the group consisting of

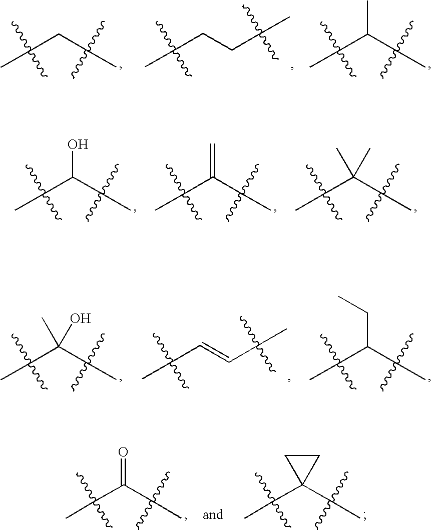

R¹ is furyl, oxazolyl, thiazolyl, pyridyl, pyrimadyl, isoxazolyl, pyrrolyl, imidazoyl, or pyridazyl, any of which may be substituted with one substituent selected from the group consisting of Cl, Br, F, OH, CN, C₍₁₋₄₎alkyl, CHF₂, CF₃, cyclopropyl, and OC₍₁₋₄₎alkyl;

R² is pyrimadyl, isoxazolyl, pyrrolyl, imidazoyl, furyl, oxazolyl, pyridyl or pyridazyl, any of which may be substituted with one substituent selected from the group consisting of Cl, Br, F, OH, C₍₁₋₄₎alkyl, CHF₂, CF₃, and OC₍₁₋₄₎alkyl;

and solvates, hydrates, tautomers and pharmaceutically acceptable salts thereof.

In another embodiment of the invention:

X is selected from the group consisting of

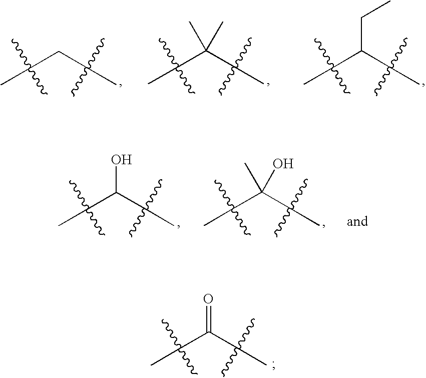

R¹ is furyl, oxazolyl, thiazolyl, pyridyl, or pyridazyl, any of which may be substituted with one substituent selected from the group consisting of Cl, Br, F, OH, CN, C₍₁₋₄₎alkyl, CHF₂, CF₃, cyclopropyl, and OC₍₁₋₄₎alkyl;

R² is pyridyl or pyridazyl, either of which may be substituted with one substituent selected from the group consisting of Cl, Br, F, OH, C₍₁₋₄₎alkyl, CHF₂, CF₃, and OC₍₁₋₄₎alkyl;

and solvates, hydrates, tautomers and pharmaceutically acceptable salts thereof.

In another embodiment of the invention:

X is selected from the group consisting of

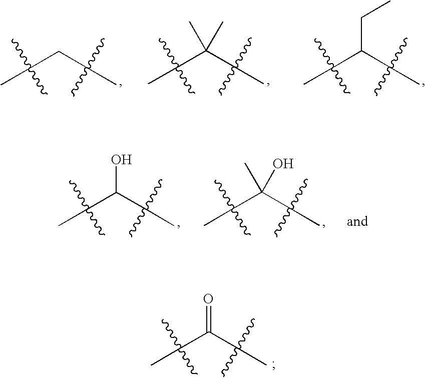

R¹ is furyl, oxazolyl, thiazolyl, pyridyl, or pyridazyl, any of which may be substituted with one substituent selected from the group consisting of CN, CH₃, CHF₂, cyclopropyl, and OCH₃;

R² is pyridyl or pyridazyl, either of which may be substituted with one substituent selected from the group consisting of Cl, Br, F, OH, and OCH₃;

and solvates, hydrates, tautomers and pharmaceutically acceptable salts thereof.

In another embodiment of the invention:

X is selected from the group consisting of

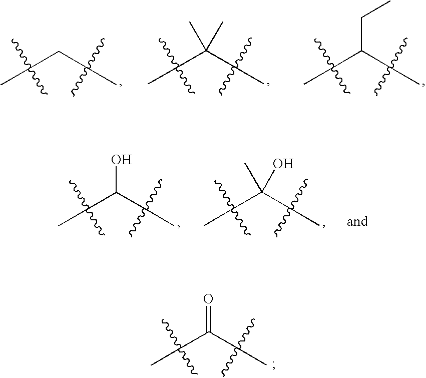

R¹ is selected from the group consisting of:

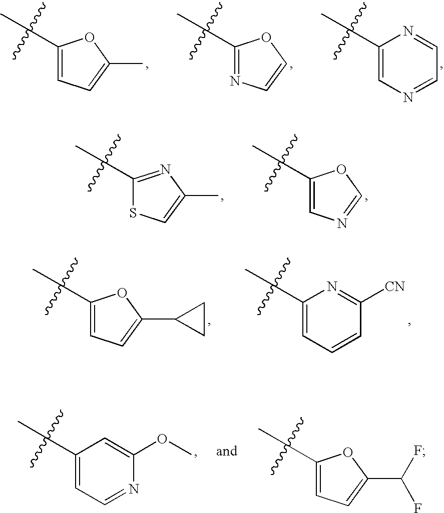

R² is selected from the group consisting of:

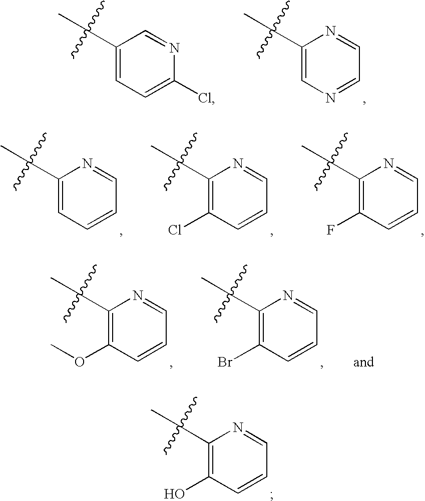

and solvates, hydrates, tautomers and pharmaceutically acceptable salts thereof.

Another embodiment of the invention is a compound selected from the group consisting of:

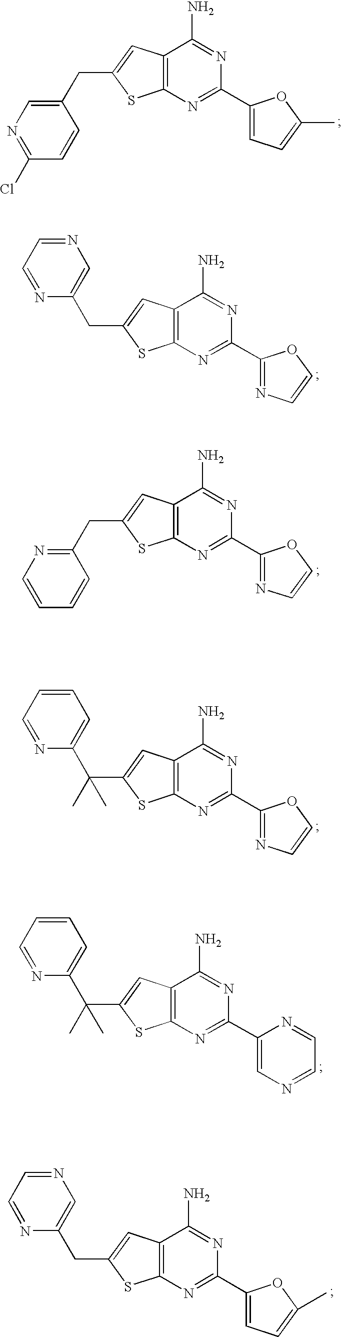

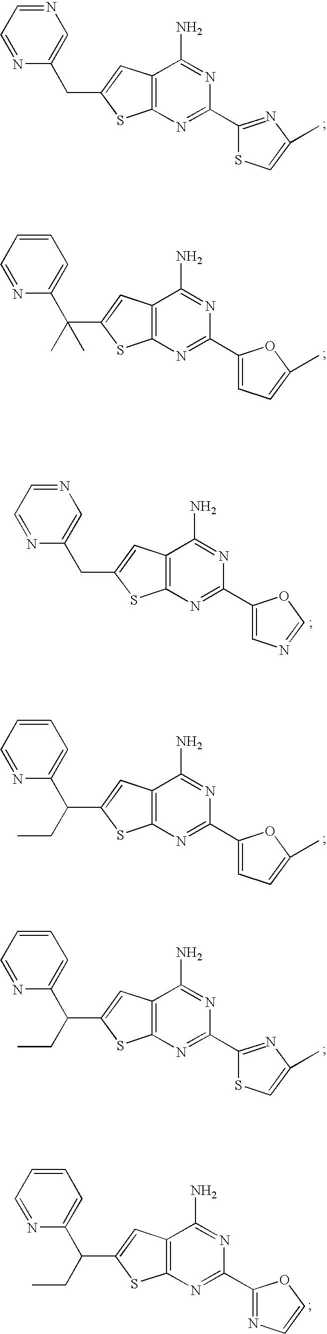

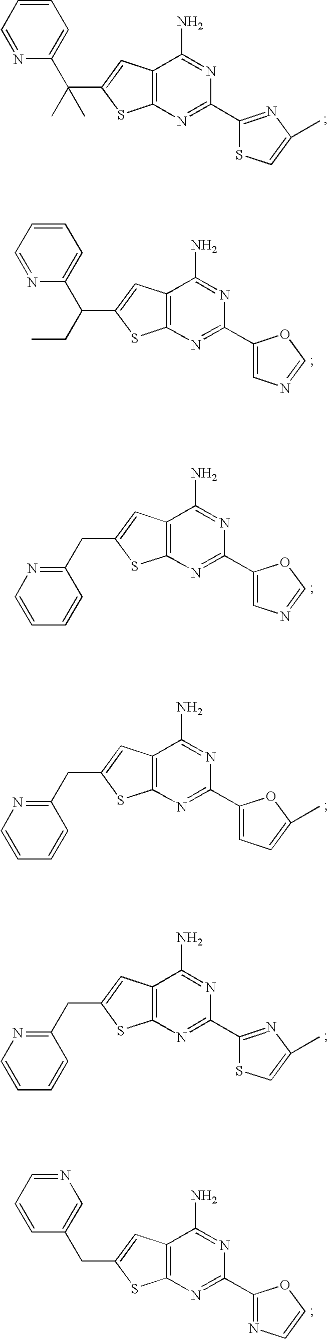

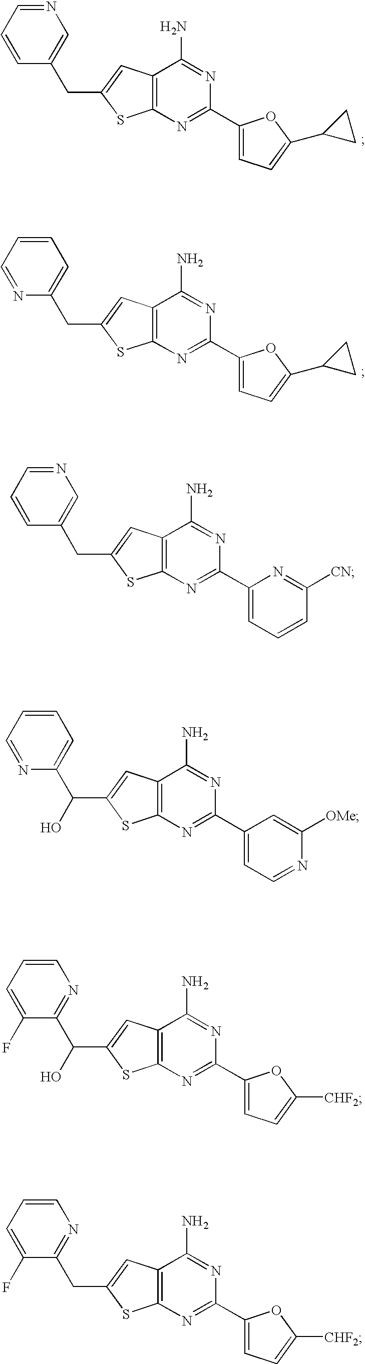

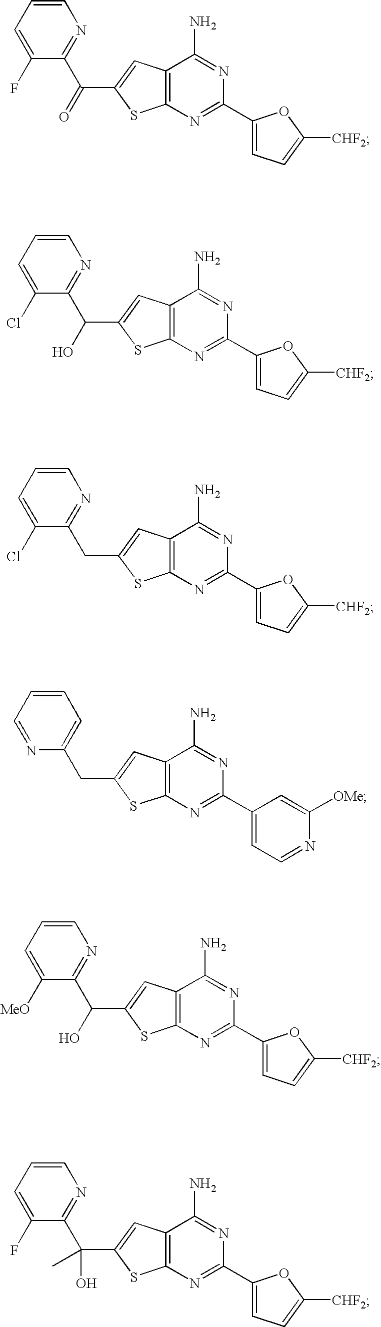

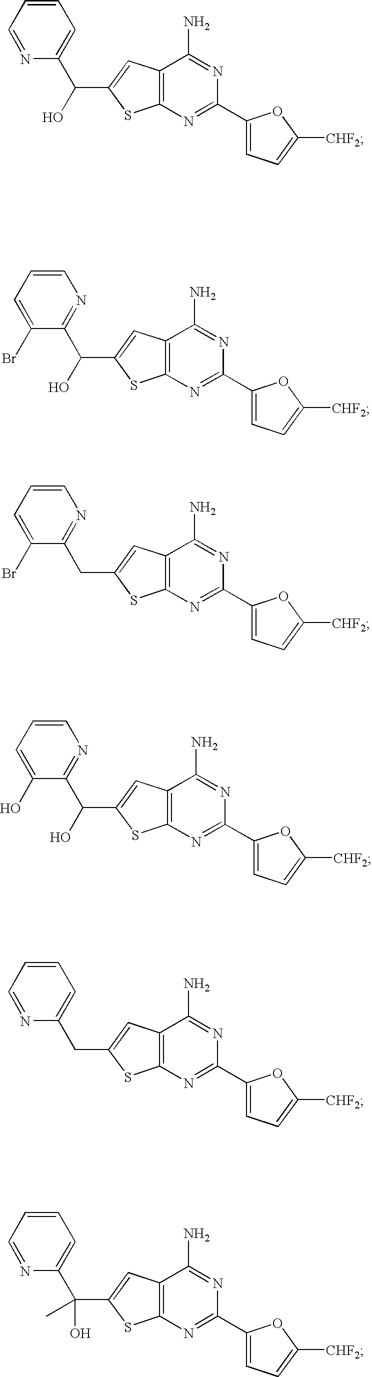

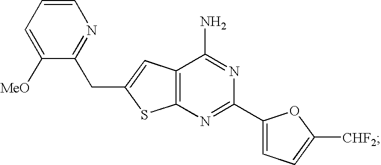

and solvates, hydrates, tautomers, and pharmaceutically acceptable salts thereof.

This invention further provides a method of treating a subject having a condition ameliorated by antagonizing Adenosine A2a receptors, which comprises administering to the subject a therapeutically effective dose of a compound of Formula Z.

This invention further provides a method of preventing a disorder ameliorated by antagonizing Adenosine A2a receptors in a subject, comprising of administering to the subject a prophylactically effective dose of the compound of claim 1 either preceding or subsequent to an event anticipated to cause a disorder ameliorated by antagonizing Adenosine A2a receptors in the subject.

Compounds of Formula Z can be isolated and used as free bases. They can also be isolated and used as pharmaceutically acceptable salts.

Examples of such salts include hydrobromic, hydroiodic, hydrochloric, perchloric, sulfuric, maleic, fumaric, malic, tartaric, citric, adipic, benzoic, mandelic, methanesulfonic, hydroethanesulfonic, benzenesulfonic, oxalic, palmoic, 2 naphthalenesulfonic, p-toluenesulfonic, cyclohexanesulfamic and saccharic.

This invention also provides a pharmaceutical composition comprising a compound of Formula Z and a pharmaceutically acceptable carrier.

Pharmaceutically acceptable carriers are well known to those skilled in the art and include, but are not limited to, from about 0.01 to about 0.1 M and preferably 0.05 M phosphate buyer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a condition ameliorated by antagonizing Adenosine A2a receptors, which comprises administering to the subject a therapeutically effective dose of a compound of Formula Z.

In one embodiment, the disorder is a neurodegenerative or movement disorder. Examples of disorders treatable by the instant pharmaceutical composition include, without limitation, Parkinson's Disease, Huntington's Disease, Multiple System Atrophy, Corticobasal Degeneration, Alzheimer's Disease, and Senile Dementia.

In one preferred embodiment, the disorder is Parkinson's disease.

As used herein, the term “subject” includes, without limitation, any animal or artificially modified animal having a disorder ameliorated by antagonizing adenosine A2a receptors. In a preferred embodiment, the subject is a human.

Administering the instant pharmaceutical composition can be effected or performed using any of the various methods known to those skilled in the art. Compounds of Formula Z can be administered, for example, intravenously, intramuscularly, orally and subcutaneously. In the preferred embodiment, the instant pharmaceutical composition is administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for the instant pharmaceutical composition. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of a compound of Formula Z. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0,ug/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The invention also provides a method of treating addiction in a mammal, comprising administering a therapeutically effective dose of a compound of Formula Z.

The invention also provides a method of treating ADHD in a mammal, comprising administering a therapeutically effective dose of a compound of Formula Z.

The invention also provides a method of treating depression in a mammal, comprising administering a therapeutically effective dose of a compound of Formula Z.

The invention also provides a method of treating anxiety in a mammal, comprising administering a therapeutically effective dose of a compound of Formula Z.

Definitions:

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “heteroaryl” refers to a radical derived by the removal of one hydrogen atom from a ring carbon atom of a heteroaromatic ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

Abbreviations:

Herein and throughout this application, the following abbreviations may be used.
Cy cyclohexyl
DMF dimethylformamide
DMSO dimethylsulfoxide
Et ethyl
EtOAc ethyl acetate
KOtBu potassium tert-butoxide
Me methyl
NBS N-bromo succinimide
OAc acetate
Pd(dppf)Cl₂ [1,1′-Bis(diphenylphosphino)ferrocene]dichloropalladium (II)
py pyridine
THF tetrahydrofuran
Xantphos 9,9-Dimethyl-4,5-bis(diphenylphosphino)xanthene

General Schemes:

Compounds of Formula Z can be prepared by methods known to those who are skilled in the art. The following reaction schemes are only meant to represent examples of the invention and are in no way meant to be a limit of the invention.

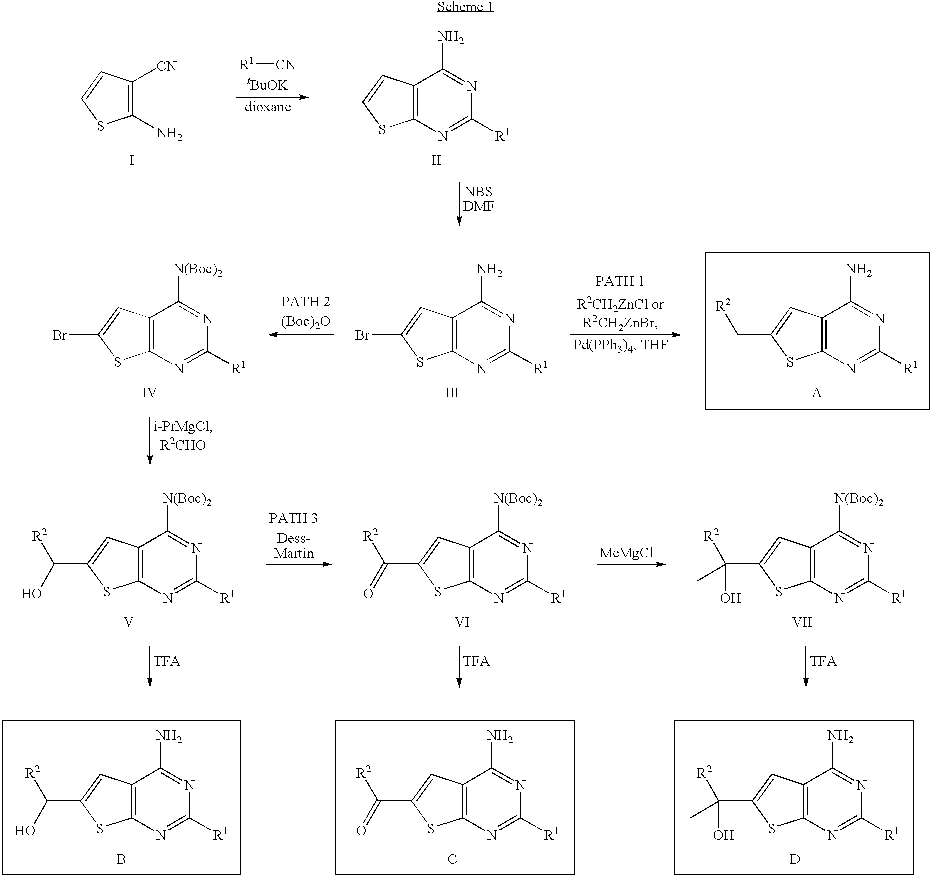

Scheme 1 illustrates the synthetic routes (Paths 1, 2 and 3) leading to compounds of Formula Z (A, B, C, and D). Starting with 2-amino-3-cyanothiophene I and following the path indicated by the arrows, condensation under basic conditions with R¹—CN, where R¹ is as defined in Formula Z, affords the aminopyrimidine II. The aminopyrimidine II is reacted with N-bromosuccinimide (NBS), to give the bromothiophene III. Following path 1 bromothiophene III is reacted with R²CH₂ZnCl or R²CH₂ZnBr, where R² is as defined in Formula Z, in the presence of a palladium catalyst to afford compounds of Formula Z, where X is CH₂ (A). Following path 2 bromothiophene III is reacted with di-tert-butyldicarbonate [(Boc)₂O] in the presence of 4-dimethylamino pyridine (DMAP) to give IV that undergoes a metal-halogen exchange and is reacted with R²CHO, where R² is as defined in Formula Z to give compounds V that are deprotected to give compounds where X is

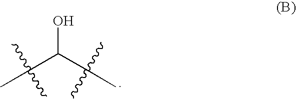

Following path 3, alcohol V is oxidized with Dess-Martin periodinane to give VI that can be deprotected to give compounds of Formula Z, where X

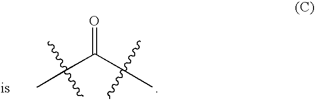

Ketones VI are reacted with methylmagnesium chloride, followed by TFA mediated BOC removal, to afford compounds of Formula Z, where X is

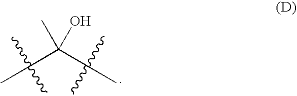

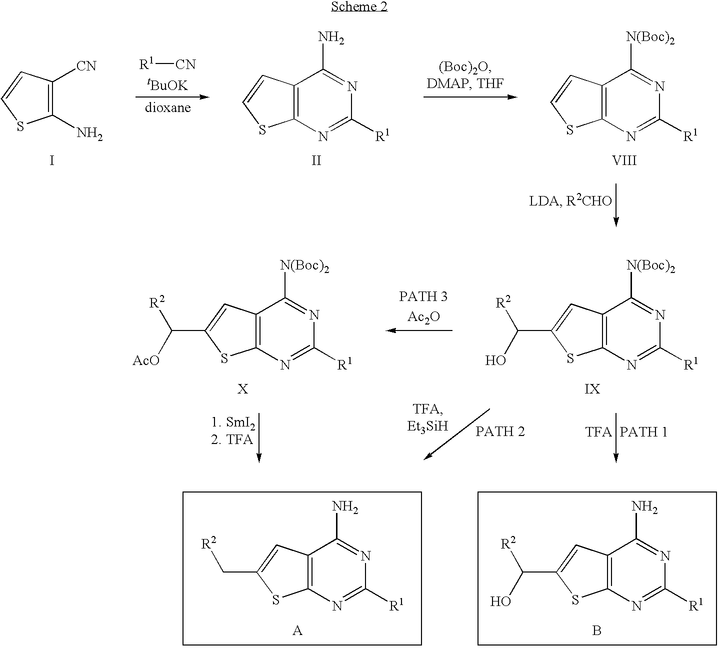

Scheme 2 illustrates the synthetic routes (Paths 1, 2 and 3) leading to compounds of Formula Z (A and B). Starting with 2-amino-3cyanothiophene I and following the path indicated by the arrows, condensation under basic conditions with R¹—CN, where R¹ is as defined in Formula Z, affords the aminopyrimidine II. The aminopyrimidine II is reacted with di-tert-butyldicarbonate [(Boc)₂O] in the presence of 4-dimethylamino pyridine (DMAP) to give the corresponding protected amine IV. The thiophene IV is deprotonated with lithium diisopropylamide (LDA) and reacted with R²CHO, where R² is as defined in Formula Z, to give an intermediate alcohol IX. Following path 1, IX is deprotected with TFA to give compounds of Formula B. Following path 2, IX is reacted with triethylsilane in TFA to give compounds of Formula A. Following path 3, IX is reacted with acetic anhydride to give the corresponding acetate X that is reduced with samarium iodide (SmI₂) followed by TFA deprotection to gives compounds of Formula A.

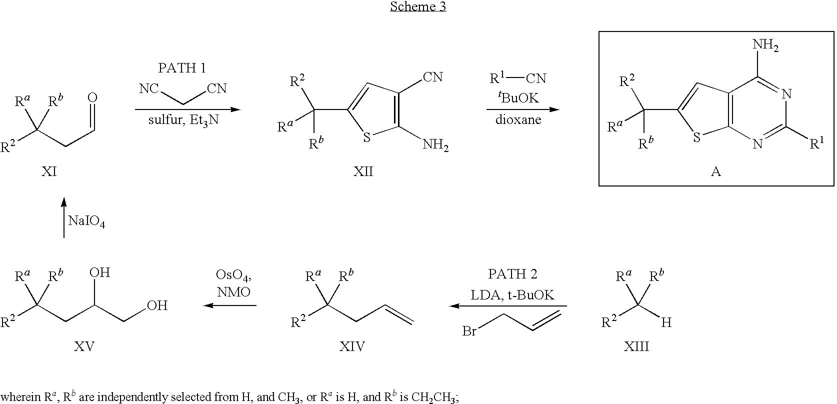

Scheme 3 illustrates the synthetic routes (Paths 1 and 2) leading to compounds of Formula A and alkyl substituted compounds of Formula A. Starting with aldehyde XI, where R² is as defined in Formula Z, reaction with malononitrile and elemental sulfur under basic conditions gives the thiophene XII. The thiophene XII is condensed under basic conditions with R¹—CN, where R¹ is as defined in Formula Z, to afford compounds of Formula Z where X is CR^{a}R^{b} and R^{a} and R^{b} are independently H, Me, or Et. Alternatively, aldehydes that are not commercially available can be synthesized following path 2 using XIII where R² is as defined in Formula Z, is reacted with allyl bromide under basic conditions to give XIV. Alkene XIV is dihydroxylated using osmium tetroxide in the presence of NMO to give diol XV. Oxidative cleavage of XV with sodium periodate affords aldehydes XI that can follow the arrows from path 1 to afford compounds of Formula A and alkyl substituted compounds of Formula A.

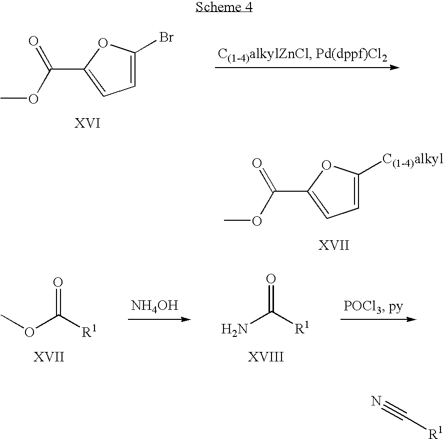

Scheme 4 illustrates the synthetic route to compounds of Formula R¹—CN, where R¹ is a C₍₁₋₄₎alkyl substituted furan. Scheme 4 also illustrates how any R¹—CO₂CH₃ may be converted into R¹—CN. Bromofuran XVI can react with alkylzinc reagents in the presence of a palladium catalyst to give XVII. Ester XVII (or any R¹—CO₂CH₃) is reacted with ammonium hydroxide to give the corresponding amide XVIII. Dehydration of the amide is accomplished using POCl₃ in pyridine to give the desired heterocyclic nitrile R¹—CN.

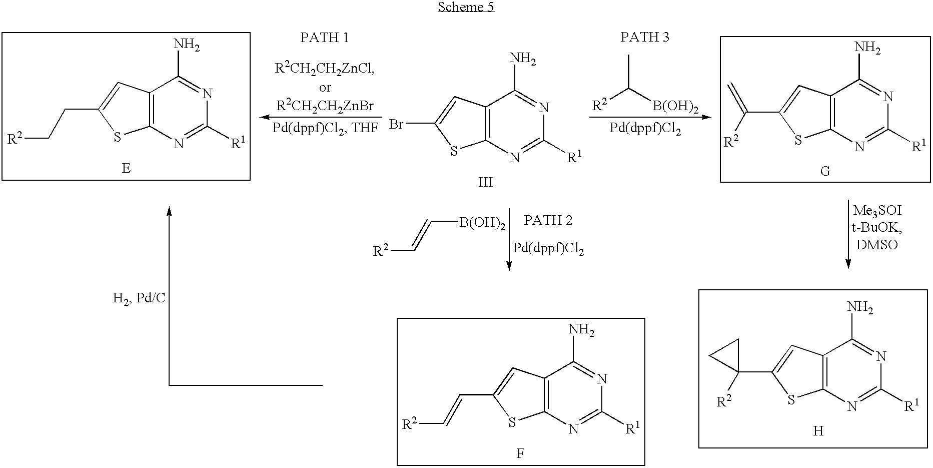

Scheme 5 illustrates the synthetic routes (Paths 1, 2 and 3) leading to compounds of Formula Z (E, F, G, and H). Following path 1, bromothiophene III is reacted with R²CH₂CH₂ZnCl or R²CH₂CH₂ZnBr, where R² is as defined in Formula Z, in the presence of a palladium catalyst to afford compounds of Formula Z, where X is CH₂CH₂ (E). Alternatively, compounds of Formula F can be reduced by hydrogenation to give compounds of Formula Z, where X is

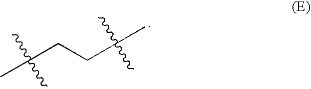

Following path 2 bromothiophene III is reacted with R²CHCHB(OH)₂, where R² is as defined in Formula Z, in the presence of a palladium catalyst to give compounds of Formula Z, where X is

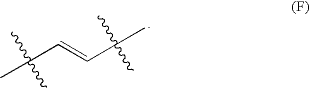

Following path 3 bromothiophene III is reacted with R²C(CH₂)B(OH)₂, where R² is as defined in Formula Z, in the presence of palladium to give compounds of Formula Z where X is

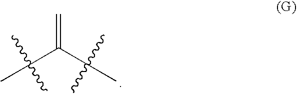

Compounds of Formula G are reacted with trimethylsufoxonium iodide under basic conditions to afford compounds of Formula Z, where X is

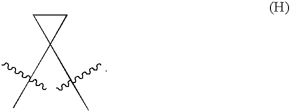

### EXAMPLES

### Example 1

### 6-(6-Chloro-pyridin-3-ylmethyl)-2-(5-methyl-furan-2-yl)-thieno[2,3-d]pyrimidin-4-ylamine

### Example 1

### Step a

### 2-(5-Methyl-furan-2-yl)-thieno[2,3-d]pyrimidin-4-ylamine

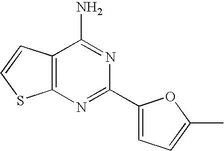

Solid t-BuOK (904 mg, 8.1 mmol) was added to a dioxane suspension (20 mL) of 2-amino-thiophene-3-carbonitrile (5.0 g, 40.3 mmol) and 5-methyl-furan-2-carbonitrile (4.5 g, 40.3 mmol) and the mixture was immersed into a 130° C. oil bath. After 10 min the flask was removed from the oil bath, diluted with THF, filtered and dry packed onto silica gel. Column chromatography gave 5.8 g of 2-(5-methyl-furan-2-yl)-thieno[2,3-d]pyrimidin-4-ylamine.

### Example 1

### Step b

### 6-Bromo-2-(5-methyl-furan-2-yl)-thieno[2,3-d]pyrimidin-4-ylamine

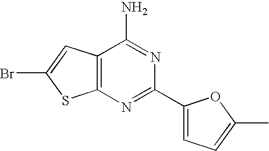

Solid NBS (4.7 g, 26.4 mmol) was added to a THF solution (100 mL) of 2-(5-methyl-furan-2-yl)-thieno[2,3-d]pyrimidin-4-ylamine (5.8 g, 25.1 mmol). After 2 h the mixture was diluted with EtOAc and washed consecutively with saturated aqueous NaHCO₃, 1 M aqueous Na₂S₂O₃, and brine. The organic layer was dried (Na₂SO₄) and dry packed onto silica gel. Column chromatography gave 6.3 g of 6-bromo-2-(5-methyl-furan-2-yl)-thieno[2,3-d]pyrimidin-4-ylamine.

### Example 1

### Step c

### 6-(6-Chloro-pyridin-3-ylmethyl)-2-(5-methyl-furan-2-yl)-thieno[2,3-d]pyrimidin-4-ylamine

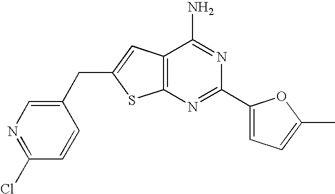

A 0.5 M THF solution of (6-chloro-3-pyridyl)methylzinc chloride (2.1 mL, 1.06 mmol) was added to a THF solution (3 mL) of 6-bromo-2-(5-methyl-furan-2-yl)-thieno[2,3-d]pyrimidin-4-ylamine (110 mg, 0.35 mmol) and Pd(dppf)Cl₂ (29 mg, 0.04 mmol) and the mixture was heated to reflux. After 3 h the mixture was diluted with EtOAc, washed with water then brine, dried (Na₂SO₄), and dry packed onto silica gel. Column chromatography gave 43 mg of the title compound. ¹H NMR (Acetone, 400 MHz): δ=7.31-7.37 (m, 4H), 7.22-7.31 (m, 1H), 7.20 (d, J=1.3 Hz, 1H), 7.02 (d, J=3.0 Hz, 1H), 6.72 (br. s., 2H), 6.15-6.20 (m, 1H), 4.23 (s, 2H), 2.36 ppm (s, 3H); MS m/e 322 (M+H).

### Example 2

### 2-Oxazol-2-yl-6-pyrazin-2-ylmethyl-thieno[2,3-d]pyrimidin-4-ylamine

### Example 2

### Step a

### 2-But-3-enyl-pyrazine

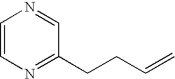

A 2.5 M hexanes solution of n-BuLi (18.0 mL, 45 mmol) was added to a −78° C. THF solution (60 mL) of t-BuOK (5.1 g, 45 mmol) and diisopropylamine (6.3 mL, 45 mmol). After 5 min at −78° C. the yellow mixture was warmed to −40° C. Neat methylpyrazine (2.7 mL, 30 mmol) was added and the mixture rapidly turned dark red. After 30 min at −40° C. the mixture was cooled to −78° C. and neat allyl bromide (7.6 mL, 90 mmol) was added. After 30 min at −78° C. water was added and the mixture was partially concentrated to remove volatile organics. The resulting mixture was extracted with dichloromethane and the combined organics were dried (Na₂SO₄), concentrated, and purified via column chromatography to give 2.2 g of 2-but-3-enyl-pyrazine.

### Example 2

### Step b

### 4-Pyrazin-2-yl-butane-1,2-diol

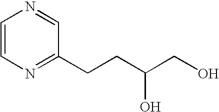

Osmium tetroxide (2.5 wt. % solution in t-BuOH, 4.0 mL, 0.32 mmol) was added to a 0° C. t-BuOH (30 mL)/water (30 mL) of 2-but-3-enyl-pyrazine (2.1 g, 15.8 mmol) and N-methyl morpholine N-oxide (2.0 g, 17.4 mmol) and the mixture was allowed to warm to rt overnight. TLC analysis indicated a low level of conversion, so an additional 8 mL of OsO₄ was added and the reaction mixture was stirred for 1 d. Conversion improved, but was still incomplete by TLC analysis; 0.5 equiv N-methyl morpholine N-oxide (925 mg) and 1.0 equiv pyridine (1.28 mL) were added, and the mixture was stirred for 2 h. A solution of 24 g Na₂SO₃ in 96 mL water was added, and the mixture was partially concentrated to remove volatile organics. The remaining aqueous solution was saturated with sodium chloride and was exhaustively extracted with ethyl acetate. The organic extracts were dried (Na₂SO₄), concentrated, and was purified by column chromatography to give 1.7 g of the title compound.

### Example 2

### Step c

### 3-Pyrazin-2-yl-propionaldehyde

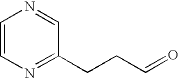

An aqueous solution of sodium periodate (0.65 M, 20 mL, 13 mmol, 1.3 equiv) was added to a suspension of silica gel (20 g) in dichloromethane (160 mL). A CH₂Cl₂ solution (10 mL) of 4-pyrazin-2-yl-butane-1,2-diol (1.7 g, 10.1 mmol) was then added. After 2 h the resulting white slurry was vacuum filtered and washed with CH₂Cl₂. The filtrate was dried (Na₂SO₄) and concentrated to give 1.1 g of the title compound that was used without further purification.

### Example 2

### Step d

### 2-Amino-5-pyrazin-2-ylmethyl-thiophene-3-carbonitrile

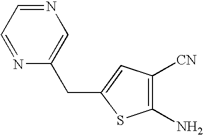

Solid elemental sulfur (257 mg, 8.0 mmol) was added to a 0° C. DMF solution (2 mL) of 3-pyrazin-2-yl-propionaldehyde (1.1 g, 8.0 mmol) and Et₃N (0.67 mL, 4.8 mmol). After 1 h, the solution was cooled to 0° C. and solid malononitrile (529 mg, 8.0 mmol) was added and stirred overnight. The mixture was partitioned between EtOAc and saturated aqueous sodium chloride, and the aqueous phase was extracted with EtOAc. The combined organic extracts were dried (Na₂SO₄), concentrated, and purified by column chromatography to give 555 mg of the title compound. ¹H NMR (CHLOROFORM-d, 300 MHz): δ (ppm) 8.44-8.59 (m, 3H), 6.54 (s, 1H), 4.73 (br. s., 2H), 4.12 (s, 2H)

### Example 2

### Step e

### Oxazole-2-carboxylic acid amide

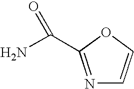

Oxazole-2-carboxylic acid ethyl ester (1.6 g, 11.4 mmol) was suspended in concentrated NH₄OH (32 mL) and stirred vigorously. After 26 h the precipitate was collected by vacuum filtration, affording 1.1 g of the title compound that was used without further purification.

### Example 2

### Step f

### Oxazole-2-carbonitrile

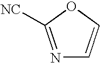

Neat POCl₃ (1.12 mL, 12.3 mmol) was added to a pyridine solution (17 mL) of oxazole-2-carboxylic acid amide (982 mg, 8.8 mmol). After 4 h the mixture was cooled to 0° C. and taken to pH 3 with concentrated aqueous HCl. The aqueous mixture was extracted with Et₂O and the combined extracts were washed with water then brine, dried (Mg₂SO₄), concentrated and used without further purification to give 478 mg of 5-cyclopropyl-furan-2-carbonitrile. The residue contained water, and was therefore dissolved in CH₂Cl₂, dried (Na₂SO₄), and concentrated to give 573 mg of the title compound that was used without further purification.

### Example 2

### Step g

### 2-Oxazol-2-yl-6-pyrazin-2-ylmethyl-thieno[2,3-d]pyrimidin-4-ylamine

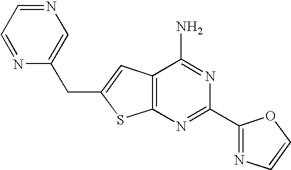

Solid t-BuOK (7 mg, 0.06 mmol) was added to a dioxane suspension (0.20 mL) of 2-amino-5-pyrazin-2-ylmethyl-thiophene-3-carbonitrile (68 mg, 0.32 mmol) and oxazole-2-carbonitrile (33 mg, 0.35 mmol) and the mixture was heated by microwave irradiation (150° C., 10 min, 300 W). The reaction mixture was diluted with dichloromethane and methanol, dry packed onto silica gel, and purified via column chromatography to give 77 mg of the title compound. ¹H NMR (DMSO-d₆, 300 MHz): δ (ppm) 8.76 (s, 1H), 8.65 (s, 1H), 8.59 (s, 1H), 8.26 (s, 1H), 7.75 (br s, 2H), 7.42 (s, 1H), 7.39 (s, 1H), 4.47 (s, 2H); MS m/e 311 (M+H).

### Example 3

### 2-Oxazol-2-yl-6-pyridin-2-ylmethyl-thieno[2,3-d]pyrimidin-4-ylamine hydrochloride

### Example 3

### Step a

### 3-Pyridin-2-yl-propionaldehyde

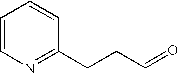

A CH₂Cl₂ solution (9 mL) of DMSO (3.7 mL, 52.5 mmol) was added to a −78° C. CH₂Cl₂ solution (20 mL) of oxalyl chloride (2.3 mL, 26.2 mmol)). After 10 min at −78° C. a CH₂Cl₂ solution (20 mL) of 2-pyridinepropanol (3.00 g, 21.9 mmol) was added. After 15 min at −78° C. neat triethylamine (15.2 mL, 109.3 mmol) was added, the mixture was stirred for 5 min at −78° C., then allowed to warm to room temperature and stirred for an additional 40 min. Water was added to the black suspension and the resulting mixture was extracted with CH₂Cl₂. The combined organic extracts were dried (Na₂SO₄), concentrated, and purified by column chromatography to give 1.2 g of the title compound.

### Example 3

### Step b

### 2-Amino-5-pyridin-2-ylmethyl-thiophene-3-carbonitrile

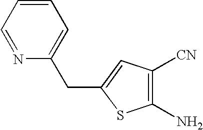

Solid elemental sulfur (239 mg, 7.5 mmol) was added to a 0° C. DMF solution (2 mL) of 3-pyridin-2-yl-propionaldehyde (1.2 g, 9.0 mmol) and Et₃N (0.62 mL, 4.5 mmol). After 50 min, the solution was cooled to 0° C. and solid malononitrile (493 mg, 7.5 mmol) was added and the mixture warmed to rt. After 40 min the mixture was partitioned between EtOAc and water, and the aqueous phase was extracted with EtOAc. The combined organic extracts were dried (Na₂SO₄), concentrated, and purified by column chromatography to give 540 mg of the title compound. ¹H NMR (300 MHz, CHLOROFORM-d) δ ppm 8.56 (d, J=4.1 Hz, 1H), 7.64 (td, J=7.6, 1.7 Hz, 1H), 7.13-7.24 (m, 2H), 6.50 (s, 1H), 4.65 (br. s., 2H), 4.09 (s, 2H).

### Example 3

### Step c

### 2-Oxazol-2-yl-6-pyridin-2-ylmethyl-thieno[2,3-d]pyrimidin-4-ylamine hydrochloride

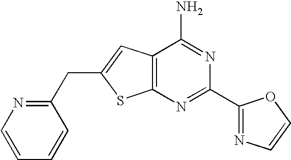

Solid t-BuOK (9 mg, 0.08 mmol) was added to a dioxane suspension (0.20 mL) of 2-amino-5-pyridin-2-ylmethyl-thiophene-3-carbonitrile (87 mg, 0.40 mmol) and oxazole-2-carbonitrile (46 mg, 0.49 mmol, prepared as an intermediate in Example 2) and the mixture was heated by microwave irradiation (130° C., 10 min, 300 W). The reaction mixture was diluted with dichloromethane and methanol, dry packed onto silica gel, and purified via column chromatography to give 107 mg of the title compound. The free base was dissolved in CH₂Cl₂ containing a minimal amount of methanol to achieve solution and the solution was added to 1 M HCl in Et₂O. The precipitated hydrochloride salt was collected by vacuum filtration to give 112 mg of the title compound. ¹H NMR (300 MHz, DMSO-D6) δ ppm 8.89 (d, J=4.9 Hz, 1H), 8.53 (t, J=7.7 Hz, 1H), 8.31 (s, 1H), 7.92-8.18 (m, 4H), 7.51 (s, 1H), 7.48 (s, 1H), 4.80 (s, 2H); MS m/e 310 (M+H).

### Example 4

### 6-(1-Methyl-1-pyridin-2-yl-ethyl)-2-oxazol-2-yl-thieno[2,3-d]pyrimidin-4-ylamine

### Example 4

### Step a

### 2-(1,1-Dimethyl-but-3-enyl)-pyridine

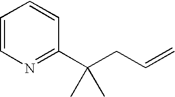

A 2.5 M hexanes solution of n-BuLi (18.0 mL, 45 mmol) was added to a −78° C. THF solution (60 mL) of t-BuOK (5.1 g, 45 mmol) and diisopropylamine (6.3 mL, 45 mmol). After 5 min at −78° C. the yellow mixture was warmed to −40° C. After 15 min, neat 2-isopropylpyridine (3.87 mL, 30 mmol) was added and the mixture rapidly turned dark red. After 30 min at −40° C. the mixture was cooled to −78° C. and neat allyl bromide (7.6 mL, 90 mmol) was added. After 30 min at −78° C. water was added and the mixture was partially concentrated to remove volatile organics. The resulting mixture was extracted with dichloromethane and the combined organics were dried (Na₂SO₄), concentrated, and purified via column chromatography to give 4.3 g of 2-(1,1-dimethyl-but-3-enyl)-pyridine.

### Example 4

### Step b

### 4-Methyl-4-pyridin-2-yl-pentane-1,2-diol

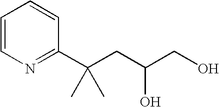

Osmium tetroxide (2.5 wt. % solution in t-BuOH, 13.4 mL, 1.1 mmol) was added to a 0° C. t-BuOH (40 mL)/water (40 mL) of 2-(1,1-dimethyl-but-3-enyl)-pyridine (3.5 g, 21.4 mmol) and N-methyl morpholine N-oxide (2.8 g, 23.6 mmol) and the mixture was allowed to warm to rt. After 3 h solid Na₂SO₃ (32 g) was added portionwise and the resulting suspension was stirred for 1 h. The mixture was partitioned between water and EtOAc and the aqueous phase was extracted with EtOAc. The combined organic extracts were dried (Na₂SO₄), concentrated, and was purified by column chromatography to give 3.9 g of the title compound.

### Example 4

### Step c

### 3-Methyl-3-pyridin-2-yl-butyraldehyde

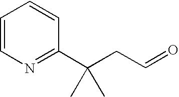

An aqueous solution of sodium periodate (0.65 M, 20 mL, 13 mmol) was added to a suspension of silica gel (20 g) in dichloromethane (160 mL). A CH₂Cl₂ solution (10 mL) solution of 4-methyl-4-pyridin-2-yl-pentane-1,2-diol (2.0 g, 10.0 mmol) was then added. After 1.5 h the resulting white slurry was vacuum filtered and washed with CH₂Cl₂. The filtrate was dried (Na₂SO₄) and concentrated to give 682 mg of the title compound.

### Example 4

### Step d

### 2-Amino-5-(1-methyl-1-pyridin-2-yl-ethyl)-thiophene-3-carbonitrile

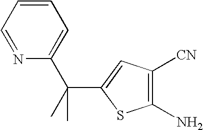

Solid elemental sulfur (110 mg, 3.4 mmol) was added to a 0° C. DMF solution (1 mL) of 3-methyl-3-pyridin-2-yl-butyraldehyde (671 mg, 4.1 mmol) and Et₃N (0.29 mL, 2.1 mmol). After 50 min, the solution was cooled to 0° C. and solid malononitrile (226 mg, 3.4 mmol) was added and stirred overnight. The mixture was partitioned between EtOAc and saturated aqueous sodium chloride, and the aqueous phase was extracted with EtOAc. The combined organic extracts were dried (Na₂SO₄), concentrated, and purified by column chromatography to give 430 mg of the title compound. ¹H NMR (CHLOROFORM-d, 300 MHz): ¹H NMR (CHLOROFORM-d, 300 MHz): δ (ppm) 8.57 (d, J=4.9 Hz, 1H), 7.61 (td, J=7.8, 2.1 Hz, 1H), 7.25-7.28 (m, 1H, obscured by CHCl₃ peak), 7.24 (dt, J=7.9, 1.1, 1H), 7.14 (ddd, J=7.5, 4.9, 1.1 Hz, 1H), 6.49 (s, 1H), 4.63 (br. s., 2H), 1.73 (s, 6H).

### Example 4

### Step e

### 6-(1-Methyl-1-pyridin-2-yl-ethyl)-2-oxazol-2-yl-thieno[2,3-d]pyrimidin-4-ylamine

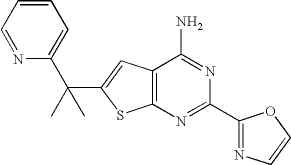

Solid t-BuOK (6 mg, 0.05 mmol) was added to a dioxane suspension (0.20 mL) of 2-amino-5-(1-methyl-1-pyridin-2-yl-ethyl)-thiophene-3-carbonitrile (63 mg, 0.26 mmol) and oxazole-2-carbonitrile (27 mg, 0.28 mmol, prepared as an intermediate in Example 2) and the mixture was heated by microwave irradiation (130° C., 10 min, 300 W). The reaction mixture was diluted with dichloromethane and methanol, dry packed onto silica gel, and purified via column chromatography to give 44 mg of the title compound. ¹H NMR (DMSO-d₆, 300 MHz): δ (ppm) 8.57 (ddd, J=4.7, 1.1, 0.9 Hz, 1H), 8.25 (s, 1H), 7.69-7.80 (m, 3H), 7.53 (s, 1H), 7.42 (s, 1H), 7.40 (d, J=7.9 Hz, 1H), 7.24-7.29 (m, 1H), 1.83 (s, 6H); MS m/e 338 (M+H).

### Example 5

### 6-(1-Methyl-1-pyridin-2-yl-ethyl)-2-pyrazin-2-yl-thieno[2,3-d]pyrimidin-4-ylamine

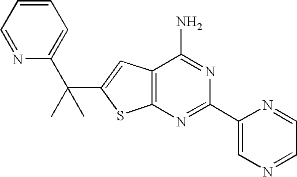

The title compound was prepared using pyrazinecarbonitrile in place of oxazole-2-carbonitrile as described in Example 4. ¹H NMR (DMSO-d₆, 300 MHz): δ (ppm) 9.45 (d, J=1.1 Hz, 1H), 8.72-8.75 (m, 1H), 8.70 (d, J=2.3 Hz, 1H), 8.55-8.59 (m, 1H), 7.76 (td, J=7.7, 1.8 Hz, 1H), 7.68 (br s, 2H), 7.54 (s, 1H), 7.40 (d, J=7.9 Hz, 1H), 7.27 (dd, J=7.4, 4.8 Hz, 1H), 1.84 (s, 6H); MS m/e 349 (M+H).

### Example 6

### 2-(5-Methyl-furan-2-yl)-6-pyrazin-2-ylmethyl-thieno[2,3-d]pyrimidin-4-ylamine

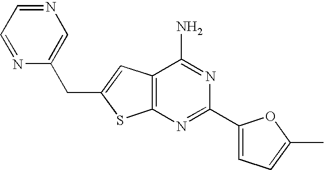

The title compound was prepared using 5-methyl-2-furonitrile in place of oxazole-2-carbonitrile as described in Example 2. ¹H NMR (DMSO-d₆, 300 MHz): δ (ppm) 8.74 (s, 1H), 8.63 (s, 1H), 8.58 (s, 1H), 7.48 (br s, 2H), 7.29 (s, 1H), 7.00 (d, J=3.0 Hz, 1H), 6.25 (d, J=3.0 Hz, 1H), 4.41 (s, 2H), 2.35 (s, 3H); MS m/e 324 (M+H).

### Example 7

### 2-(4-Methyl-thiazol-2-yl)-6-pyrazin-2-ylmethyl-thieno[2,3-d]pyrimidin-4-ylamine

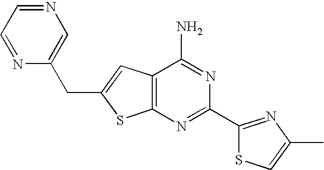

The title compound was prepared using 4-methylthiazole-2-carbonitrile in place of oxazole-2-carbonitrile as described in Example 2. ¹H NMR (DMSO-d₆, 300 MHz): δ (ppm) 8.75 (s, 1H), 8.63-8.66 (m, 1H), 8.59 (d, J=2.6 Hz, 1H), 7.69 (br s, 2H), 7.40 (s, 1H), 7.37 (s, 1H), 4.45 (s, 2H), 2.43 (s, 3H); MS m/e 341 (M+H).

### Example 8

### 2-(5-Methyl-furan-2-yl)-6-(1-methyl-1-pyridin-2-yl-ethyl)-thieno[2,3-d]pyrimidin-4-ylamine

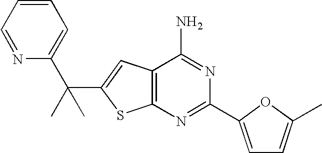

The title compound was prepared using 5-methyl-2-furonitrile in place of oxazole-2-carbonitrile as described in Example 4. ¹H NMR (DMSO-d₆, 300 MHz): δ (ppm) 8.56 (d, J=4.9 Hz, 1H), 7.74 (dt, J=7.7, 1.9 Hz, 1H), 7.47 (br s, 2H), 7.43 (s, 1H), 7.36 (d, J=7.9 Hz, 1H), 7.25 (dd, J=7.5, 4.9 Hz, 1H), 6.99 (d, J=3.0 Hz, 1H), 6.24 (d, J=3.0 Hz, 1H), 2.35 (s, 3H), 1.80 (s, 6H); MS m/e 351 (M+H).

### Example 9

### 2-Oxazol-5-yl-6-pyrazin-2-ylmethyl-thieno[2,3-d]pyrimidin-4-ylamine

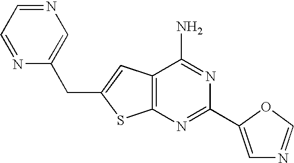

The title compound was prepared using oxazole-5-carbonitrile in place of oxazole-2-carbonitrile as described in Example 2. ¹H NMR (DMSO-d₆, 300 MHz): δ (ppm) 8.74 (s, 1H), 8.64 (s, 1H), 8.58 (d, J=2.6 Hz, 1H), 8.51 (s, 1H), 7.73 (s, 1H), 7.65 (br s, 2H), 7.34 (s, 1H), 4.44 (s, 2H); MS m/e 311 (M+H).

### Example 10

### (±)-2-(5-Methyl-furan-2-yl)-6-(1-pyridin-2-yl-propyl)-thieno[2,3-d]pyrimidin-4-ylamine hydrochloride

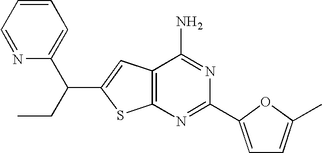

The title compound was prepared using 2-n-propylpyridine and 5-methyl-2-furonitrile in place of 2-isopropylpyridine and oxazole-2-carbonitrile, respectively as described in Example 4. ¹H NMR (DMSO-d₆, 300 MHz): δ (ppm) 8.77 (d, J=5.3 Hz, 1H), 8.25 (t, J=7.5 Hz, 1H), 7.81 (d, J=7.9 Hz, 1H), 7.65-7.73 (m, 2H), 7.49 (d, J=3.0 Hz, 1H), 6.42 (d, J=3.4 Hz, 1H), 4.68 (t, J=7.5 Hz, 1H), 2.41 (s, 3H), 2.14-2.31 (m, 2H), 0.91 (t, J=7.2 Hz, 3H); MS m/e 351 (M+H).

### Example 11

### (±)-2-(4-Methyl-thiazol-2-yl)-6-(1-pyridin-2-yl-propyl)-thieno[2,3-d]pyrimidin-4-ylamine hydrochloride

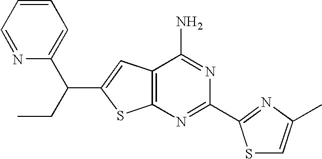

The title compound was prepared using 2-n-propylpyridine and 4-methylthiazole-2-carbonitrile in place of 2-isopropylpyridine and oxazole-2-carbonitrile, respectively, as described in Example 4. ¹H NMR (DMSO-d₆, 300 MHz): δ (ppm) 8.75 (d, J=4.9 Hz, 1H), 8.20 (t, J=7.5 Hz, 1H), 7.78 (d, J=7.5 Hz, 1H), 7.61-7.69 (m, 2H), 7.57 (s, 1H), 4.65 (t, J=7.7 Hz, 1H), 2.14-2.31 (m, 2H), 0.91 (t, J=7.2 Hz, 3H); MS m/e 368 (M+H).

### Example 12

### (±)-2-Oxazol-2-yl-6-(1-pyridin-2-yl-propyl)-thieno[2,3-d]pyrimidin-4-ylamine hydrochloride

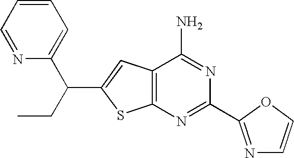

The title compound was prepared using 2-n-propylpyridine in place of 2-isopropylpyridine as described in Example 4. ¹H NMR (DMSO-d₆, 300 MHz): δ (ppm) 8.81 (d, J=4.9 Hz, 1H), 8.29-8.40 (m, 2H), 7.90 (d, J=7.9 Hz, 1H), 7.78 (t, J=6.4 Hz, 1H), 7.62 (s, 1H), 7.47 (s, 1H), 4.75 (t, J=7.5 Hz, 1H), 2.18-2.34 (m, 2H), 0.92 (t, J=7.3 Hz, 3H); MS m/e 338 (M+H).

### Example 13

### 6-(1-Methyl-1-pyridin-2-yl-ethyl)-2-(4-methyl-thiazol-2-yl)-thieno[2,3-d]pyrimidin-4-ylamine

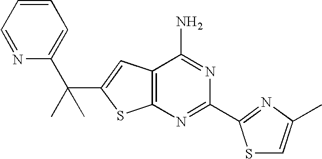

The title compound was prepared using 4-methylthiazole-2-carbonitrile in place of oxazole-2-carbonitrile as described in Example 4. ¹H NMR (DMSO-d₆, 300 MHz): δ (ppm) 8.57 (d, J=3.8 Hz, 1H), 7.76 (td, J=7.7, 1.9 Hz, 1H), 7.67 (br s, 2H), 7.51 (s, 1H), 7.39 (t, J=4.0 Hz, 2H), 7.27 (dd, J=7.0, 5.1 Hz, 1H), 2.43 (s, 3H), 1.83 (s, 6H); MS m/e 368 (M+H).

### Example 14

### (±)-2-Oxazol-5-yl-6-(1-pyridin-2-yl-propyl)-thieno[2,3-d]pyrimidin-4-ylamine hydrochloride

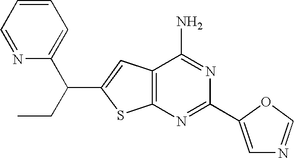

The title compound was prepared using 2-n-propylpyridine and oxazole-5-carbonitrile in place of 2-isopropylpyridine and oxazole-2-carbonitrile, respectively, as described in Example 4. ¹H NMR (DMSO-d₆, 300 MHz): δ(ppm) 8.78 (d, J=4.9 Hz, 1H), 8.54 (s, 1H), 8.28 (t, J=8.1 Hz, 1H), 7.67-7.90 (m, 4H), 7.53 (s, 1H), 4.65 (t, J=7.5 Hz, 1H), 2.16-2.32 (m, 2H), 0.91 (t, J=7.3 Hz, 3H); MS m/e 338 (M+H).

### Example 15

### 2-Oxazol-5-yl-6-pyridin-2-ylmethyl-thieno[2,3-d]pyrimidin-4-ylamine

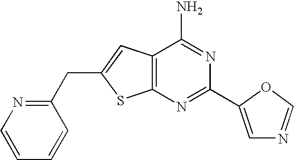

The title compound was prepared using oxazole-5-carbonitrile in place of oxazole-2-carbonitrile as described in Example 3. 1H NMR (300 MHz, CHLOROFORM-D) δ ppm 8.59 (d, J=4.1 Hz, 1H), 8.00 (s, 1H), 7.82 (s, 1H), 7.67 (td, J=7.6, 1.7 Hz, 1H), 7.18-7.31 (m, 2H), 6.93 (s, 1H), 5.44 (br s, 2H), 4.37 (s, 2H); MS m/e 310 (M+H).

### Example 16

### 2-(5-Methyl-furan-2-yl)-6-pyridin-2-ylmethyl-thieno[2,3-d]pyrimidin-4-ylamine

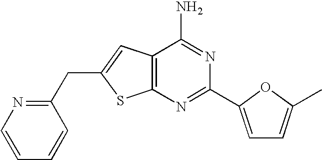

The title compound was prepared using 5-methyl-2-furonitrile in place of oxazole-2-carbonitrile as described in Example 3. 1H NMR (300 MHz, CHLOROFORM-D) δ ppm 8.54 (d, J=3.8 Hz, 1H), 7.62 (td, J=7.6, 1.7 Hz, 1H), 7.23 (d, J=7.9 Hz, 1H), 7.13-7.17 (m, 2H), 6.86 (s, 1H), 6.12 (d, J=2.3 Hz, 1H), 5.61 (s, 2H), 4.31 (s, 2H), 2.41 (s, 3H); MS m/e 322 (M+H).

### Example 17

### 2-(4-Methyl-thiazol-2-yl)-6-pyridin-2-ylmethyl-thieno[2,3-d]pyrimidin-4-ylamine

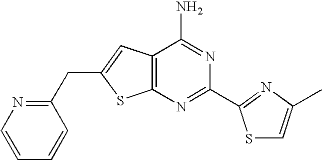

The title compound was prepared using 4-methylthiazole-2-carbonitrile in place of oxazole-2-carbonitrile as described in Example 3. 1H NMR (300 MHz, CHLOROFORM-D) δ ppm 8.59 (d, J=4.1 Hz, 1H), 7.67 (td, J=7.7, 1.9 Hz, 1H), 7.17-7.29 (m, 2H), 7.02 (s, 1H), 6.93 (s, 1H), 5.46 (br s, 2H), 4.37 (s, 2H), 2.56 (s, 3H); MS m/e 340 (M+H).

### Example 18

### 2-Oxazol-2-yl-6-pyridin-3-ylmethyl-thieno[2,3-d]pyrimidin-4-ylamine

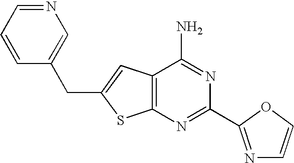

The title compound was prepared using 3-pyridinepropanol in place of 2-pyridinepropanol as described in Example 3. 1H NMR (300 MHz, MeOD) δ ppm 8.55 (d, J=1.9 Hz, 1H), 8.46 (dd, J=4.7, 1.3 Hz, 1H), 8.09 (s, 1H), 7.82 (d, J=7.9 Hz, 1H), 7.43 (dd, J=7.7, 5.1 Hz, 1H), 7.40 (s, 1H), 7.24 (s, 1H), 4.33 (s, 2H); MS m/e 310 (M+H).

### Example 19

### 2-(5-Cyclopropyl-furan-2-yl)-6-pyridin-3-ylmethyl-thieno[2,3-d]pyrimidin-4-ylamine

### Example 19

### Step a

### 5-Cyclopropyl-furan-2-carboxylic acid methyl ester

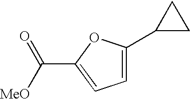

Solid cyclopropylboronic acid (575 mg, 6.7 mmol) was added to a toluene (22 mL)/water (1.1 mL) solution of 5-bromo-furan-2-carboxylic acid methyl ester (980 mg, 4.8 mmol), Pd(OAc)₂ (54 mg, 0.2 mmol), P(Cy)₃ (135 mg, 0.5 mmol), and K₃PO₄ (3.6 g, 16.8 mmol). The resulting mixture was heated to 90° C. After 5 h the mixture was cooled, filtered and extracted with EtOAc. The combined organic extracts were washed with water and brine, dried (Na₂SO₄), concentrated and purified via column chromatography to give 650 mg of 5-cyclopropyl-furan-2-carboxylic acid methyl ester.

### Example 19

### Step b

### 5-Cyclopropyl-furan-2-carboxylic acid amide

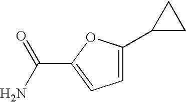

5-cyclopropyl-furan-2-carboxylic acid methyl ester (650 mg, 3.9 mmol) was suspended in concentrated NH₄OH (20 mL) and stirred vigorously. After 16 h the mixture was diluted with water and the aqueous phase was extracted with EtOAc. The combined organic extracts were washed with water and brine, dried (Na₂SO₄), concentrated and used without further purification to give 550 mg of 5-cyclopropyl-furan-2-carboxylic acid amide.

### Example 19

### Step c

### 5-Cyclopropyl-furan-2-carbonitrile

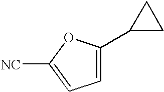

Neat POCl₃ (0.48 mL, 5.1 mmol) was added to a pyridine solution (9 mL) of 5-cyclopropyl-furan-2-carboxylic acid amide (550 mg, 3.6 mmol). After 2 h the mixture was cooled to 0° C. and taken to pH 4.5 with concentrated aqueous HCl. The aqueous mixture was extracted with Et₂O and the combined extracts were washed with brine, dried (Na₂SO₄), concentrated and used without further purification to give 478 mg of 5-cyclopropyl-furan-2-carbonitrile.

### Example 19

### Step d

### 2-(5-Cyclopropyl-furan-2-yl)-6-pyridin-3-ylmethyl-thieno[2,3-d]pyrimidin-4-ylamine

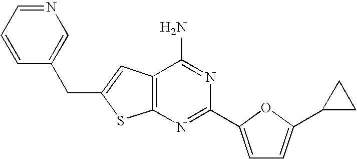

The title compound was prepared using 3-pyridinepropanol and 5-cyclopropyl-furan-2-carbonitrile in place of 2-pyridinepropanol and oxazole-2-carbonitrile, respectively as described in Example 3. ¹H NMR (DMSO-d₆, 300 MHz): δ=8.57 (d, J=1.9 Hz, 1H), 8.49 (dd, J=4.9, 1.5 Hz, 1H), 7.68-7.77 (m, 1H), 7.45 (s, 1H), 7.23 (s, 1H), 6.99 (d, J=3.4 Hz, 1H), 6.20 (d, J=3.4 Hz, 1H), 4.24 (s, 2H), 3.33 (s, 2H), 2.01 (s, 1H), 0.89-0.99 (m, 2H), 0.71-0.80 ppm (m, 2H); MS m/e 349 (M+H).

### Example 20

### 6-Benzyl-2-(5-cyclopropyl-furan-2-yl)-thieno[2,3-d]pyrimidin-4-ylamine hydrochloride

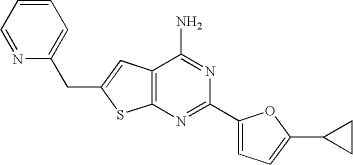

The title compound was prepared using 5-cyclopropyl-furan-2-carbonitrile in place of and oxazole-2-carbonitrile as described in Example 3. ¹H NMR (DMSO-d₆, 300 MHz): δ=8.84 (br. s., 1H), 8.38 (br. s., 1H), 7.87 (d, J=8.3 Hz, 2H), 7.51 (s, 1H), 7.39-7.44 (m, 1H), 6.40 (d, J=3.0 Hz, 1H), 4.69 (s, 2H), 2.08 (t, J=5.1 Hz, 1H), 1.24 (br. s., 2H), 0.86 ppm (s, 2H); MS m/e 349 (M+H).

### Example 21

### 6-(4-Amino-6-pyridin-3-ylmethyl-thieno[2,3-d]pyrimidin-2-yl)-pyridine-2-carbonitrile

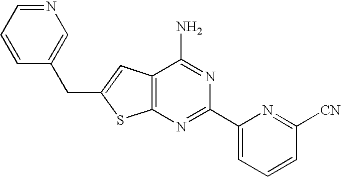

The title compound was prepared using 3-pyridinepropanol and pyridine-2,6-dicarbonitrile in place of 2-pyridinepropanol and oxazole-2-carbonitrile, respectively as described in Example 3. ¹H NMR (CHLOROFORM-d, 300 MHz): δ=8.02 (d, J=6.8 Hz, 2H), 7.45-7.71 (m, 4H), 7.19 (s, 1H), 6.99 (dd, J=8.7, 1.9 Hz, 1H), 3.83 (s, 2H), 3.3 ppm (s, 2H); MS m/e 345 (M+H).

### Example 22

### [4-Amino-2-(2-methoxy-pyridin-4-yl)-thieno[2,3-d]pyrimidin-6-yl]-phenyl-methanol

### Example 22

### Step a

### 2-(2-Methoxy-pyridin-4-yl)-thieno[2,3-d]pyrimidin-4-ylamine

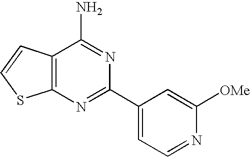

The title compound was prepared using 2-methoxy-isonicotinonitrile and in place of 5-methyl-furan-2-carbonitrile as described in Example 1.

### Example 22

### Step b

### [2-(2-Methoxy-pyridin-4-yl)-thieno[2,3-d]pyrimidin-4-yl]-bis-carbamic acid tert-butyl ester

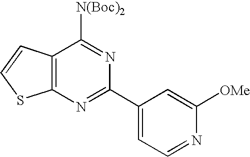

Solid DMAP (100 mg, 0.82 mmol) was added to a THF solution (20 mL) of 2-(2-methoxy-pyridin-4-yl)-thieno[2,3-d]pyrimidin-4-ylamine (2.0 g, 8.0 mmol) and (Boc)₂O (4.4 g, 20.2 mmol). After 2 h the mixture was concentrated in vacuo, and the resulting solid was diluted with CH₂Cl₂, filtered, and the filtrate was concentrated and purified by column chromatography to give 3.0 g of the title compound.

### Example 22

### Step c

### [6-(Hydroxy-pyridin-2-yl-methyl)-2-(2-methoxy-pyridin-4-yl)-thieno[2,3-d]pyrimidin-4-yl]-bis-carbamic acid tert-butyl ester

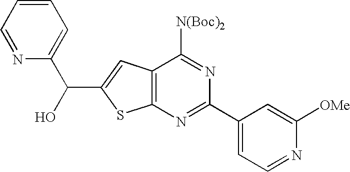

A 1.8 M LDA solution (0.54 mL, 0.97 mmol) was added to a −78° C. THF solution (3.5 mL) of [2-(2-methoxy-pyridin-4-yl)-thieno[2,3-d]pyrimidin-4-yl]-bis-carbamic acid tert-butyl ester (400 mg, 0.87 mmol). After 8 min, neat pyridine-2-carbaldehyde (140 mg, 1.31 mmol) was added and the mixture was allowed to warm to −20° C. over 40 min. Saturated aqueous NH₄Cl was added and the layers were separated. The aqueous layer was extracted with CH₂Cl₂ and the combined organics were dried (Na₂SO₄), concentrated, and purified by column chromatography to give 200 mg of the title compound.

### Example 22

### Step d

### [4-Amino-2-(2-methoxy-pyridin-4-yl)-thieno[2,3-d]pyrimidin-6-yl]-phenyl-methanol

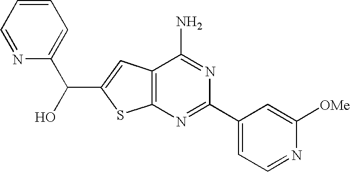

Neat TFA (0.15 mL) was added to a CH₂Cl₂ solution (0.4 mL) of [6-(hydroxy-phenyl-methyl)-2-(2-methoxy-pyridin-4-yl)-thieno[2,3-d]pyrimidin-4-yl]-bis-carbamic acid tert-butyl ester (40 mg). After 1 h the mixture was concentrated and the resulting solid was partitioned between CH₂Cl₂ and saturated aqueous NaHCO₃. The organic phase was separated, dried (Na₂SO₄), and concentrated to provide 37 mg of the title compound. ¹H NMR (300 MHz, Acetone-d₆) δ=8.56 (d, J=4.9 Hz, 1H), 8.22 (d, J=5.3 Hz, 1H), 7.79-7.92 (m, 2H), 7.62-7.72 (m, 2H), 7.49 (s, 1H), 7.32 (dd, J=4.9, 7.5 Hz, 1H), 6.99 (br. s., 2H), 6.13 (d, J=4.5 Hz, 1H), 5.92 (br. s., 1H), 3.92 (s, 3H); MS m/e 366 (M+H).

### Example 23

### [4-Amino-2-(5-difluoromethyl-furan-2-yl)-thieno[2,3-d]pyrimidin-6-yl]-(3-fluoro-pyridin-2-yl)-methanol

### Example 23

### Step a

### 5-Difluoromethyl-furan-2-carbonitrile

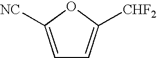

To a solution of Et₂NSF₃ (2.8 mL, 21.4 mmol) and CH₂Cl₂ (10 mL) at 4° C. was added a solution of 5-formyl-furan-2-carbonitrile (2.44 g, 20.2 mmol; W. Hoyle and G. P. Roberts, J. Med. Chem. 1973, 16, 709) in CH₂Cl₂ (10 mL). After 30 min at 4° C., saturated aqueous NaHCO₃ was added, the layers were separated and the aqueous layer was extracted with CH₂Cl₂. The combined organics were dried (Na₂SO₄) and concentrated to give 2.15 g of 5-difluoromethyl-furan-2-carbonitrile that was used without further purification.

### Example 23

### Step b

### 2-(5-Difluoromethyl-furan-2-yl)-thieno[2,3-d]pyrimidin-4-ylamine

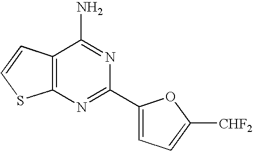

Solid t-BuONa (280 mg, 2.9 mmol) was added to a dioxane solution (8 mL) of 5-difluoromethyl-furan-2-carbonitrile (2.1 g, 14.7 mmol) and 2-amino-thiophene-3-carbonitrile (1.8 g, 14.7 mmol) and the mixture immediately became very hot, some solid was formed, and stirring became difficult. An additional 4 mL of dioxane was added and the mixture was stirred. After 1.5 h the mixture was diluted with THF and dry packed onto silica gel. Column chromatography gave 2.9 g of the title compound.

### Example 23

### Step c

### 6-Bromo-2-(5-difluoromethyl-furan-2-yl)-thieno[2,3-d]pyrimidin-4-ylamine

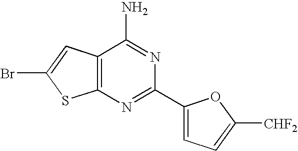

Solid NBS (3.0 g, 16.6 mmol) was added to a THF solution (110 mL) of 2-(5-difluoromethyl-furan-2-yl)-thieno[2,3-d]pyrimidin-4-ylamine (3.1 g, 11.8 mmol). After 2.5 h the mixture was dry packed onto silica gel. Column chromatography gave 4.8 g of the title compound.

### Example 23

### Step d

### [6-Bromo-2-(5-difluoromethyl-furan-2-yl)-thieno[2,3-d]pyrimidin-4-yl]-bis-carbamic acid tert-butyl ester

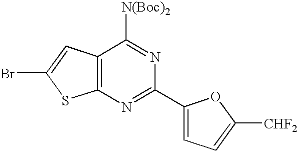

Solid DMAP (170 mg, 1.39 mmol) was added to a THF solution (130 mL) of 6-bromo-2-(5-difluoromethyl-furan-2-yl)-thieno[2,3-d]pyrimidin-4-ylamine (4.8 g, 13.9 mmol) and Boc₂O (7.93 g, 36.4 mmol). After 40 min the mixture was concentrated in vacuo and purified via column chromatography to afford 4.6 g of the title compound.

### Example 23

### Step e

### {2-(5-Difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester

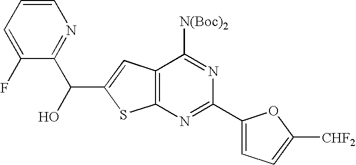

A 1.0 M THF solution of i-PrMgCl.LiCl (0.70 mL, 0.70 mmol) was added to a −78° C. THF solution (1 mL) of [6-bromo-2-(5-difluoromethyl-furan-2-yl)-thieno[2,3-d]pyrimidin-4-yl]-bis-carbamic acid tert-butyl ester (308 mg, 0.56 mmol). After 1 h at −78° C. a THF solution (0.8 mL) of 3-fluoro-pyridine-2-carbaldehyde (106 mg, 0.85 mmol) was added and the reaction mixture was warmed to −15° C. After 40 min, saturated aqueous NH₄Cl was added and the aqueous phase was extracted with CH₂Cl₂. The combined organics were dried (Na₂SO₄), concentrated, and purified via column chromatography to give 183 mg of the title compound.

### Example 23

### Step f

### [4-Amino-2-(5-difluoromethyl-furan-2-yl)-thieno[2,3-d]pyrimidin-6-yl]-(3-fluoro-pyridin-2-yl)-methanol

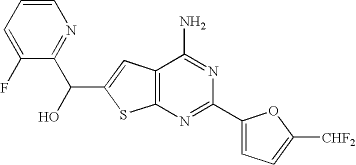

Neat TFA (0.2 mL) was added to a CH₂Cl₂ solution (0.8 mL) of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester (42 mg, 0.07 mmol). After 1 h the mixture was concentrated in vacuo and the residue was partitioned between saturated aqueous NaHCO₃ and EtOAc. The layers were separated and the organic layer was dried (Na₂SO₄) and concentrated to give 16 mg of the title compound. ¹H NMR (300 MHz, Acetone-d₆) δ=8.49 (d, J=4.5 Hz, 1H), 7.62-7.75 (m, 1H), 7.51 (dt, J=4.3, 8.4 Hz, 1H), 7.40 (s, 1H), 7.14-7.25 (m, 1H), 7.00 (t, J=53.7 Hz, 1H), 6.89-7.09 (m, 3H), 6.33 (s, 1H), 5.63 (br. s., 1H); MS m/e 393 (M+H).

### Example 24

### 2-(5-Difluoromethyl-furan-2-yl)-6-(3-fluoro-pyridin-2-ylmethyl)-thieno[2,3-d]pyrimidin-4-ylamine

### Example 24

### Step a

### Acetic acid [4-tert-butoxycarbonylamino-2-(5-difluoromethyl-furan-2-yl)-thieno[2,3-d]pyrimidin-6-yl]-(3-fluoro-pyridin-2-yl)-methyl ester

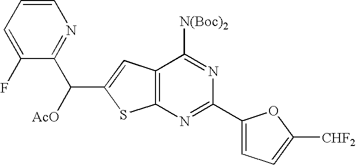

Solid DMAP (5 mg, 0.04 mmol) was added to a CH₂Cl₂ solution (1 mL) of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester (120 mg, 0.20 mmol, an intermediate prepared in Example 23), Ac₂O (50 μL, 0.53 mmol) and pyridine (0.1 mL). After 5 h the mixture was concentrated and purified by flash chromatography to give 110 mg of the title compound.

### Example 24

### Step b

### [2-(5-Difluoromethyl-furan-2-yl)-6-(3-fluoro-pyridin-2-ylmethyl)-thieno[2,3-d]pyrimidin-4-yl]-bis-carbamic acid tert-butyl ester

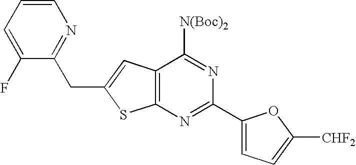

A THF solution (1.5 mL) of acetic acid [4-tert-butoxycarbonylamino-2-(5-difluoromethyl-furan-2-yl)-thieno[2,3 -d]pyrimidin-6-yl]-(3-fluoro-pyridin-2-yl)-methyl ester (100 mg, 0.16 mmol) was purged with N₂ for ˜6 min, t-BuOH (23 μL, 0.24 mmol) was added followed by a 0.1 M THF solution of SmI₂ (6.4 mL, 0.64 mmol). After stirring of the blue mixture for 25 min, the blue color changed to yellow and an additional 6.4 mL of the 0.1 M THF solution of SmI₂ was added. After 1 h saturated aqueous NH₄Cl was added and the organic layer was separated. The aqueous layer was extracted with EtOAc and the combined organics were dried (Na₂SO₄), concentrated, and purified by column chromatography to yield 58 mg of the title compound.

### Example 24

### Step c

### 2-(5-Difluoromethyl-furan-2-yl)-6-(3-fluoro-pyridin-2-ylmethyl)-thieno[2,3-d]pyrimidin-4-ylamine

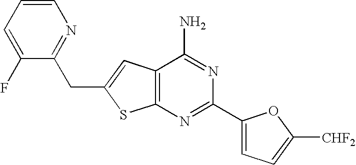

Neat TFA (0.3 mL) was added to a CH₂Cl₂ solution (1.2 mL) of [2-(5-Difluoromethyl-furan-2-yl)-6-(3-fluoro-pyridin-2-ylmethyl)-thieno[2,3 -d]pyrimidin-4-yl]-bis-carbamic acid tert-butyl ester (58 mg, 0.10 mmol). After 30 min the mixture was concentrated in vacuo and the residue was partitioned between saturated aqueous NaHCO₃ and EtOAc. The layers were separated and the organic layer was dried (Na₂SO₄) and concentrated to give 37 mg of the title compound. ¹H NMR (300 MHz, Acetone-d₆) δ=8.41 (d, J=4.9 Hz, 1H), 7.62 (t, J=9.2 Hz, 1H), 7.40 (dt, J=4.4, 8.5 Hz, 1H), 7.33 (s, 1H), 7.14-7.23 (m, 1H), 7.00 (t, J=53.9 Hz, 1H), 6.87-6.98 (m, 3H), 4.45 (d, J=1.9 Hz, 2H); MS m/e 377 (M+H).

### Example 25

### [4-Amino-2-(5-difluoromethyl-furan-2-yl)-thieno[2,3-d]pyrimidin-6-yl]-(3-fluoro-pyridin-2-yl)-methanone

### Example 25

### Step a

### [2-(5-Difluoromethyl-furan-2-yl)-6-(3-fluoro-pyridine-2-carbonyl)-thieno[2,3-d]pyrimidin-4-yl]-bis-carbamic acid tert-butyl ester

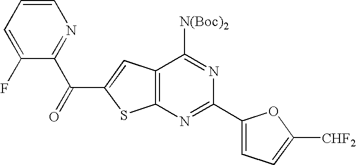

Solid Dess-Martin periodinane (90 mg, 0.21 mmol) was added to a CH₂Cl₂ solution (1.5 mL) of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester (94 mg, 0.16 mmol, an intermediate prepared in Example 23). After 1 h and saturated aqueous NaHCO₃ and saturated aqueous Na₂S₂O₃ were added. The mixture was stirred vigorously for ˜15 min and the aqueous phase was extracted with CH₂Cl₂. The combined extracts were dried (Na₂SO₄), concentrated, and purified via column chromatography to yield 71 mg of the title compound.

### Example 25

### Step b

### [4-Amino-2-(5-difluoromethyl-furan-2-yl)-thieno[2,3-d]pyrimidin-6-yl]-(3-fluoro-pyridin-2-yl)-methanone

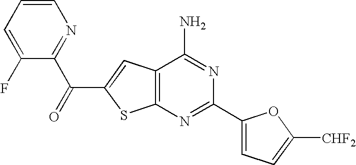

Neat TFA (0.2 mL) was added to a CH₂Cl₂ solution (0.8 mL) of [2-(5-difluoromethyl-furan-2-yl)-6-(3-fluoro-pyridine-2-carbonyl)-thieno[2,3 -d]pyrimidin-4-yl]-bis-carbamic acid tert-butyl ester (25 mg, 0.04 mmol). After 1 h the mixture was concentrated in vacuo and the residue was partitioned between saturated aqueous NaHCO₃ and EtOAc. The layers were separated and the organic layer was dried (Na₂SO₄) and concentrated to give 16 mg of the title compound. ¹H NMR (300 MHz, DMSO-d₆) δ=8.63 (d, J=4.5 Hz, 1H), 8.46 (s, 1H), 8.20 (br. s., 2H), 8.01 (t, J=9.6 Hz, 1H), 7.81 (m, 1H), 7.30 (d, J=3.4 Hz, 1H), 7.17 (t, J=52.9 Hz, 1H), 7.06-7.04 (m, 1H); MS m/e 391 (M+H).

### Example 26

### [4-Amino-2-(5-difluoromethyl-furan-2-yl)-thieno[2,3-d]pyrimidin-6-yl]-(3-chloro-pyridin-2-yl)-methanol

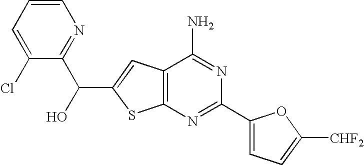

The title compound was prepared using 3-chloro-pyridine-2-carbaldehyde in place of 3-fluoro-pyridine-2-carbaldehyde as described in example 23. ¹H NMR (300 MHz, Acetone-d₆) δ=8.63 (d, J=4.5 Hz, 1H), 7.95 (d, J=8.3 Hz, 1H), 7.48 (dd, J=4.7, 7.7 Hz, 1H), 7.40 (s, 1H), 7.15-7.25 (m, 1H), 6.91-7.12 (m, 3H), 7.00 (t, J=53.7 Hz, 1H), 6.37 (d, J=6.8 Hz, 1H), 5.51-5.74 (m, 1H); MS m/e 409/411 (M+H).

### Example 27

### 6-(3-Chloro-pyridin-2-ylmethyl)-2-(5-difluoromethyl-furan-2-yl)-thieno[2,3-d]pyrimidin-4-ylamine

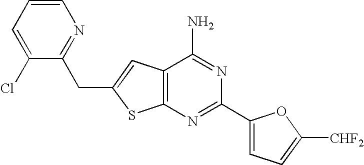

[6-[(3-Chloro-pyridin-2-yl)-hydroxy-methyl]-2-(5-difluoromethyl-furan-2-yl)-thieno[2,3 -d]pyrimidin-4-yl]-bis-carbamic acid tert-butyl ester (36 mg, 0.06 mmol, an intermediate prepared in Example 26), Et₃SiH (0.3 mL), and TFA (0.4 mL) were combined and heated at 80° C. in the microwave for 1.5 h. The mixture was concentrated in vacuo, and the residue was partitioned between EtOAc and saturated aqueous NaHCO₃. The organic phase was dried (Na₂SO₄), concentrated, and purified by preparative thin layer chromatography to yield 15 mg of the title compound. ¹H NMR (300 MHz, Acetone-d₆) δ=8.46-8.58 (m, 1H), 7.88 (dd, J=1.5, 7.9 Hz, 1H), 7.36 (dd, J=4.7, 8.1 Hz, 1H), 7.32 (s, 1H), 7.14-7.22 (m, 1H), 7.00 (t, J=53.9 Hz, 1H), 6.86-6.98 (m, 3H), 4.54 (s, 2H); MS m/e 393/395 (M+H).

### Example 28

### 2-(2-Methoxy-pyridin-4-yl)-6-pyridin-2-ylmethyl-thieno[2,3-d]pyrimidin-4-ylamine

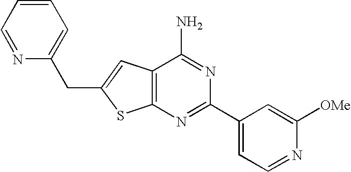

The title compound was prepared using [6-(hydroxy-phenyl-methyl)-2-(2-methoxy-pyridin-4-yl)-thieno[2,3-d]pyrimidin-4-yl]-bis-carbamic acid tert-butyl ester (an intermediate prepared in Example 22) in place of [6-[(3-chloro-pyridin-2-yl)-hydroxy-methyl]-2-(5-difluoromethyl-furan-2-yl)-thieno[2,3-d]pyrimidin-4-yl]-bis-carbamic acid tert-butyl ester as described in Example 27. ¹H NMR (300 MHz, Acetone-d₆) δ=8.56 (d, J=4.5 Hz, 1H), 8.22 (d, J=5.3 Hz, 1H), 7.87 (d, J=5.7 Hz, 1H), 7.76 (m, 1H), 7.69 (s, 1H), 7.41 (d, J=7.9 Hz, 1H), 7.35 (s, 1H), 7.21-7.31 (m, 1H), 6.93 (br. s., 2H), 4.40 (s, 2H), 3.93 (s, 3H); MS m/e 350 (M+H).

### Example 29

### [4-Amino-2-(5-difluoromethyl-furan-2-yl)-thieno[2,3-d]pyrimidin-6-yl]-(3-methoxy-pyridin-2-yl)-methanol

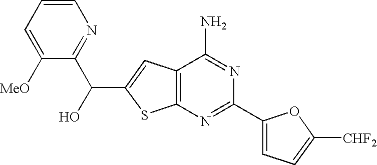

The title compound was prepared using 3-methoxy-pyridine-2-carbaldehyde in place of 3-fluoro-pyridine-2-carbaldehyde as described in example 23. ¹H NMR (300 MHz, Acetone-d₆) δ=8.20 (d, J=4.1 Hz, 1H), 7.51 (d, J=8.3 Hz, 1H), 7.33-7.46 (m, 2H), 7.08-7.22 (m, 1H), 6.99 (t, J=53.5 Hz, 1H), 6.85-6.98 (m, 2H), 6.22 (s, 1H), 3.92 (s, 3H); MS m/e 405 (M+H).

### Example 30

### 1-[4-Amino-2-(5-difluoromethyl-furan-2-yl)-thieno[2,3-d]pyrimidin-6-yl]-1-(3-fluoro-pyridin-2-yl)-ethanol

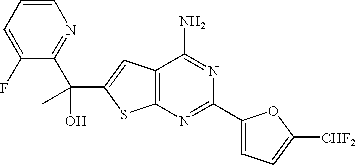

A 3.0 M THF solution of MeMgCl (0.04 mL, 0.12 mmol) was added to a 4° C. THF solution (1 mL) of [2-(5-difluoromethyl-furan-2-yl)-6-(3-fluoro-pyridine-2-carbonyl)-thieno[2,3-d]pyrimidin-4-yl]-bis-carbamic acid tert-butyl ester (44 mg, 0.07 mmol, an intermediate prepared in Example 25). After 30 min at 4° C. the mixture was warmed to rt. After 1 h at rt an additional 0.04 mL of the 3.0 M THF solution of MeMgCl was added and the mixture was stirred overnight. Saturated aqueous NH₄Cl was added and the aqueous phase was extracted with CH₂Cl₂. The combined organics were dried (Na₂SO₄), concentrated, and purified by column chromatography to yield 25 mg of white solid that was used without further purification. A solution of this solid in 0.4 mL of CH₂Cl₂ was treated with neat TFA (0.1 mL). After 1 h The mixture was concentrated in vacuo, and the residue was partitioned between EtOAc and saturated aqueous NaHCO₃. The organic phase was dried (Na₂SO₄) and concentrated to yield 19 mg of the title compound that was analytically pure. ¹H NMR (300 MHz, Acetone-d₆) δ=8.48 (d, J=4.1 Hz, 1H), 7.63-7.76 (m, 1H), 7.52-7.61 (m, 1H), 7.50 (s, 1H), 7.19 (d, J=3.4 Hz, 1H), 7.00 (t, J=53.9 Hz, 1H), 6.91-7.00 (m, 3H), 6.46 (br. s., 1H), 2.05 m/e 407 (M+H).

### Example 31

### [4-Amino-2-(5-difluoromethyl-furan-2-yl)-thieno[2,3-d]pyrimidin-6-yl]-pyridin-2-yl-methanol

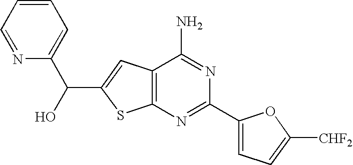

The title compound was prepared using pyridine-2-carbaldehyde in place of 3-fluoro-pyridine-2-carbaldehyde as described in Example 23. ¹H NMR (300 MHz, Acetone-d₆) δ=8.56 (d, J=3.8 Hz, 1H), 7.75-7.90 (m, 1H), 7.65 (d, J=7.9 Hz, 1H), 7.46 (s, 1H), 7.32 (dd, J=4.9, 7.5 Hz, 1H), 7.10-7.20 (m, 1H), 7.00 (t, J=53.5 Hz, 1H), 6.90-7.00 (m, 3H), 6.11 (s, 1H), 5.87 (br. s., 1H); MS m/e 375 (M+H).

### Example 32

### [4-Amino-2-(5-difluoromethyl-furan-2-yl)-thieno[2,3-d]pyrimidin-6-yl]-(3-bromo-pyridin-2-yl)-methanol

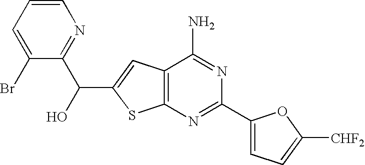

The title compound was prepared using 3-bromo-pyridine-2-carbaldehyde in place of 3-fluoro-pyridine-2-carbaldehyde as described in Example 23. ¹H NMR (300 MHz, MeOD-d₄) δ=8.81 (m, 1H), 8.52-8.72 (m, 1H), 7.63-7.92 (m, 2H), 7.46-7.63 (m, 1H), 7.05 (m, 1H), 6.92 (t, J=53.7 Hz, 1H), 6.63 (br. s., 1H); MS m/e 453/455 (M+H).

### Example 33

### 6-(3-Bromo-pyridin-2-ylmethyl)-2-(5-difluoromethyl-furan-2-yl)-thieno[2,3-d]pyrimidin-4-ylamine

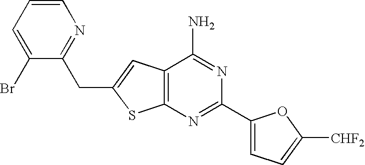

The title compound was prepared using [6-[(3-bromo-pyridin-2-yl)-hydroxy-methyl]-2-(5-difluoromethyl-furan-2-yl)-thieno[2,3-d]pyrimidin-4-yl]-bis-carbamic acid tert-butyl ester (an intermediate prepared in Example 32) in place of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester as described in Example 24. ¹H NMR (300 MHz, Acetone-d₆) δ=8.50-8.61 (m, 1H), 7.97-8.08 (m, 1H), 7.31-7.33 (m, 1H), 7.27 (dd, J=4.7, 8.1 Hz, 1H), 7.11-7.21 (m, 1H), 7.00 (t, J=53.9 Hz, 1H), 6.92-6.97 (m, 3H), 4.56 (s, 2H); MS m/e 437/439 (M+H).

### Example 34

### 2-{[4-Amino-2-(5-difluoromethyl-furan-2-yl)-thieno[2,3-d]pyrimidin-6-yl]-hydroxy-methyl}-pyridin-3-ol

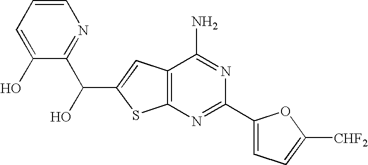

The title compound was prepared using 3-hydroxy-pyridine-2-carbaldehyde in place of 3-fluoro-pyridine-2-carbaldehyde as described in example 23. ¹H NMR (300 MHz, Acetone-d₆) δ=8.09-8.17 (m, 1H), 7.41 (s, 1H), 7.21-7.36 (m, 2H), 7.18 (d, J=3.4 Hz, 1H), 6.99 (t, J=53.9 Hz, 1H), 6.87-6.98 (m, 2H), 6.25 (s, 1H); MS m/e 391 (M+H).

### Example 35

### 2-(5-Difluoromethyl-furan-2-yl)-6-pyridin-2-ylmethyl-thieno[2,3-d]pyrimidin-4-ylamine

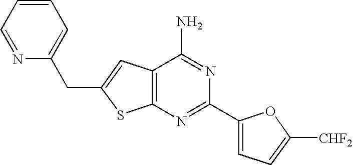

The title compound was prepared using [2-(5-difluoromethyl-furan-2-yl)-6-(hydroxy-pyridin-2-yl-methyl)-thieno[2,3-d]pyrimidin-4-yl]-bis-carbamic acid tert-butyl ester (an intermediate prepared in Example 31) in place of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester as described in Example 24. ¹H NMR (300 MHz, MeOD-d₄) δ=8.86 (d, J=5.7 Hz, 1H), 8.54-8.69 (m, 1H), 7.96-8.13 (m, 2H), 7.53-7.68 (m, 2H), 7.04-7.13 (m, 1H), 6.97 (t, J=53.6 Hz, 1H), 4.84 (s, 2H); MS m/e 359 (M+H).

### Example 36

### 1-[4-Amino-2-(5-difluoromethyl-furan-2-yl)-thieno[2,3-d]pyrimidin-6-yl]-1-pyridin-2-yl-ethanol

### Example 36

### Step a

### [2-(5-Difluoromethyl-furan-2-yl)-6-(pyridine-2-carbonyl)-thieno[2,3-d]pyrimidin-4-yl]-bis-carbamic acid tert-butyl ester

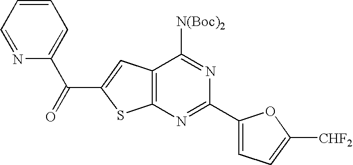

The title compound was prepared using [2-(5-difluoromethyl-furan-2-yl)-6-(hydroxy-pyridin-2-yl-methyl)-thieno[2,3-d]pyrimidin-4-yl]-bis-carbamic acid tert-butyl ester (an intermediate prepared in Example 31) in place of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester as described in Example 25.

### Example 36

### Step b

### 1-[4-Amino-2-(5-difluoromethyl-furan-2-yl)-thieno[2,3-d]pyrimidin-6-yl]-1-pyridin-2-yl-ethanol

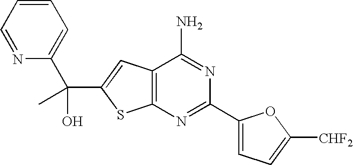

The title compound was prepared using [2-(5-difluoromethyl-furan-2-yl)-6-(pyridine-2-carbonyl)-thieno[2,3-d]pyrimidin-4-yl]-bis-carbamic acid tert-butyl ester in place of [2-(5-difluoromethyl-furan-2-yl)-6-(3 -fluoro-pyridine-2-carbonyl)-thieno[2,3 -d]pyrimidin-4-yl]-bis-carbamic acid tert-butyl ester as described in Example 30. ¹H NMR (300 MHz, Acetone-d₆) δ=8.52 (d, J=4.5 Hz, 1H), 7.79 (t, J=7.5 Hz, 1H), 7.71 (d, J=7.9 Hz, 1H), 7.48 (s, 1H), 7.27 (t, J=6.0 Hz, 1H), 7.15 (d, J=3.4 Hz, 1H), 6.96 (t, J=53.5 Hz, 1H), 6.90-6.96 (m, 3H), 1.99 (s, 3H); MS m/e 389 (M+H).

### Example 37

### 2-(5-Difluoromethyl-furan-2-yl)-6-(3-methoxy-pyridin-2-ylmethyl)-thieno[2,3-d]pyrimidin-4-ylamine

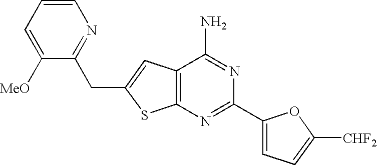

The title compound was prepared using {2-(5-difluoromethyl-furan-2-yl)-6-[hydroxy-(3-methoxy-pyridin-2-yl)-methyl]-thieno[2,3-d]pyrimidin-4-yl}-bis-carbamic acid tert-butyl ester (an intermediate prepared in Example 29) in place of {2-(5-difluoromethyl-furan-2-yl)-6-[(3-fluoro-pyridin-2-yl)-hydroxy-methyl]-thieno[2,3-d]pyrimidin-4-yl}-bis-carbamic acid tert-butyl ester as described in Example 24. ¹H NMR (300 MHz, MeOD-d₄) δ=8.42 (d, J=4.5 Hz, 1H), 8.31-8.34 (m, 1H), 7.95-8.12 (m, 1H), 7.50-7.70 (m, 2H), 7.05-7.13 (m, 1H), 6.97 (t, J=53.3 Hz, 1H), 4.74 (s, 2H), 4.16 (s, 3H); MS m/e 389 (M+H)

Biological Assays and Activity

Ligand Binding Assay for Adenosine A2a Receptor (A2A-B)

Ligand binding assay of adenosine A2a receptor was performed using plasma membrane of HEK293 cells containing human A2a adenosine receptor (PerkinElmer, RB-HA2a) and radioligand [³H]CGS21680 (PerkinElmer, NET1021). Assay was set up in 96-well polypropylene plate in total volume of 200 μL by sequentially adding 20 μL 1:20 diluted membrane, 130 μL assay buffer (50 mM Tris.HCl, pH7.4 10 mM MgCl₂, 1 mM EDTA) containing [³H] CGS21680, 50 μL diluted compound (4×) or vehicle control in assay buffer. Nonspecific binding was determined by 80 mM NECA. Reaction was carried out at room temperature for 2 hours before filtering through 96-well GF/C filter plate pre-soaked in 50 mM Tris.HCl, pH7.4 containing 0.3% polyethylenimine. Plates were then washed 5 times with cold 50 mM Tris.HCl, pH7.4, dried and sealed at the bottom. Microscintillation fluid 30 μL was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693)

Adenosine A2a Receptor Functional Assay (A2AGAL2)

To initiate the functional assay, cryopreserved CHO-K1 cells overexpressing the human adenosine A2a receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 15 nM NECA (Sigma E2387) agonist challenge (5 uL volume). A control curve of NECA, a DMSO/Media control, and a single dose of Forskolin (Sigma F3917) were also included on each plate. After additions, cell plates were allowed to incubate at 37° C., 5% CO₂, 90% Rh for 5.5-6 hours. After incubation, media was removed, and cell plates were washed 1×50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme colorimetric assay, plates were thawed out at room temperature and 20μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

Adenosine A1 Receptor Functional Assay (A1GAL2)

To initiate the functional assay, cryopreserved CHO-K1 cells overexpressing the human adenosine A1 receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech #10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 4 nM r-PIA (Sigma P4532)/1 uM Forskolin (Sigma F3917) agonist challenge (5 uL volume). A control curve of r-PIA in 1 uM Forskolin, a DMSO/Media control, and a single dose of Forskolin were also included on each plate. After additions, cell plates were allowed to incubate at 37° C., 5% CO₂, 90% Rh for 5.5-6 hours. After incubation, media was removed, and cell plates were washed 1×50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme colorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The colorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

A2a ASSAY DATA

| | | | |
|---|---|---|---|
| Example | A2AGAL2 Ki μM | A2A-B Ki μM | A1GAL2 Ki μM |

| | | | |
|---|---|---|---|
| 1 | 0.126212 | ND | 1.58708 |
| 2 | ND | ND | ND |
| 3 | 0.0066819 | ND | 0.352777 |
| 4 | ND | ND | ND |
| 5 | ND | ND | ND |
| 6 | ND | ND | ND |
| 7 | ND | ND | ND |
| 8 | ND | ND | ND |
| 9 | ND | ND | ND |
| 10 | ND | ND | ND |
| 11 | ND | ND | ND |
| 12 | ND | ND | ND |
| 13 | ND | ND | ND |
| 14 | ND | ND | ND |
| 15 | 0.00966273 | ND | 0.172147 |
| 16 | 0.00118005 | ND | 0.0299364 |
| 17 | 0.00709414 | ND | 0.100462 |
| 18 | 0.0300124 | ND | 0.489441 |
| 19 | 0.409449 | ND | 0.579162 |
| 20 | 0.129867 | ND | 0.33721 |
| 21 | 0.00525049 | ND | 0.0281838 |
| 22 | ND | ND | ND |
| 23 | 0.00377051 | ND | 0.0595388 |
| 24 | 0.00620869 | ND | 0.092619 |
| 25 | 0.0248599 | ND | 0.838301 |
| 26 | ND | ND | ND |
| 27 | ND | ND | ND |
| 28 | ND | ND | ND |
| 29 | 0.00239166 | ND | 0.0248771 |
| 30 | 0.0113684 | 0.0162817 | 0.40281 |
| 31 | 0.00481947 | ND | 0.0469029 |
| 32 | 0.0016248 | ND | 0.0235342 |
| 33 | 0.00217721 | ND | 0.0212031 |
| 34 | 0.00428845 | ND | 0.110586 |
| 35 | 0.00473151 | ND | 0.0618871 |
| 36 | 0.0359832 | ND | 0.267055 |
| 37 | 0.00228402 | ND | 0.0326137 |

ND indicates that no data was available.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

1. A compound of Formula Z 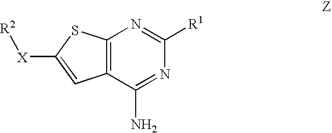
wherein
X is selected from the group consisting of 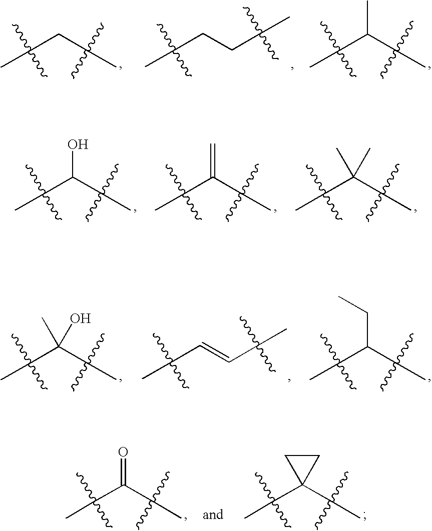
R¹ is heteroaryl which may be substituted with one substituent selected from the group consisting of Cl, Br, F, OH, CN, C₍₁₋₄₎alkyl, CHF₂, CF₃, OCF₃, cyclopropyl, and OC₍₁₋₄₎alkyl;
R² is heteroaryl wherein said heteroaryl is optionally substituted with Cl, F, Br, OC₍₁₋₄₎alkyl, OCF₃, OH, C₍₁₋₄₎alkyl, CHF₂, CF₃, OCH₂CF₃, or a ring selected from the group consisting of: 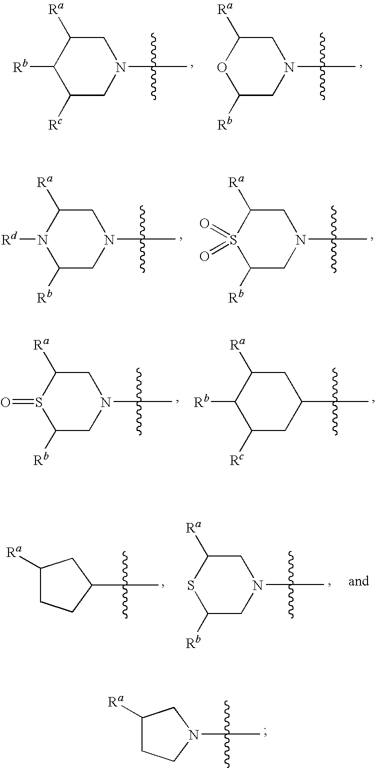
wherein R^{a}, R^{b}, and R^{c} are independently H or C₍₁₋₄₎alkyl;
R^{d} is H, —C₍₁₋₄₎alkyl, —CH₂CH₂OCH₂CH₂OCH₃, —CH₂CO₂H, —C(O)C₍₁₋₄₎alkyl, or —CH₂C(O)C₍₁₋₄₎alkyl;
and solvates, hydrates, tautomers and pharmaceutically acceptable salts thereof.

2. A compound of claim 1, wherein:
R¹ is furyl, oxazolyl, thiazolyl, pyridyl, pyrimadyl, isoxazolyl, pyrrolyl, imidazoyl, or pyridazyl, any of which may be substituted with one substituent selected from the group consisting of Cl, Br, F, OH, CN, C₍₁₋₄₎alkyl, CHF₂, CF₃, cyclopropyl, and OC₍₁₋₄₎alkyl;
R² is pyrimadyl, isoxazolyl, pyrrolyl, imidazoyl, furyl, oxazolyl, pyridyl or pyridazyl, any of which may be substituted with one substituent selected from the group consisting of Cl, Br, F, OH, C₁₋₄₎alkyl, CHF₂, CF₃, and OC₍₁₋₄₎alkyl;
and solvates, hydrates, tautomers and pharmaceutically acceptable salts thereof.

3. A compound of claim 2, wherein:
X is selected from the group consisting of 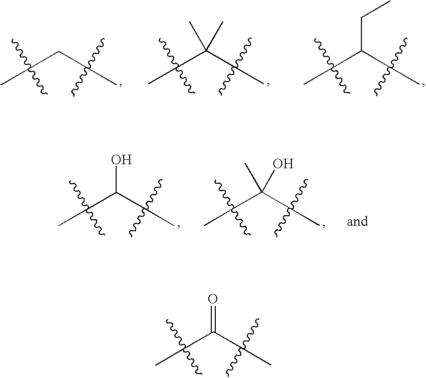
R¹ is furyl, oxazolyl, thiazolyl, pyridyl, or pyridazyl, any of which may be substituted with one substituent selected from the group consisting of Cl, Br, F, OH, CN, C₍₁₋₄₎alkyl, CHF₂, CF₃, cyclopropyl, and OC₍₁₋₄₎alkyl;
R² is pyridyl or pyridazyl, either of which may be substituted with one substituent selected from the group consisting of Cl, Br, F, OH, C₍₁₋₄₎alkyl, CHF₂, CF₃, and OC₁₋₄₎alkyl;
and solvates, hydrates, tautomers and pharmaceutically acceptable salts thereof.

4. A compound of claim 3, wherein:
R¹ is furyl, oxazolyl, thiazolyl, pyridyl, or pyridazyl, any of which may be substituted with one substituent selected from the group consisting of CN, CH₃, CHF₂, cyclopropyl, and OCH₃;
R² is pyridyl or pyridazyl, either of which may be substituted with one substituent selected from the group consisting of Cl, Br, F, OH, and OCH₃;
and solvates, hydrates, tautomers and pharmaceutically acceptable salts thereof.

5. A compound of claim 4, wherein:
R¹ is selected from the group consisting of: 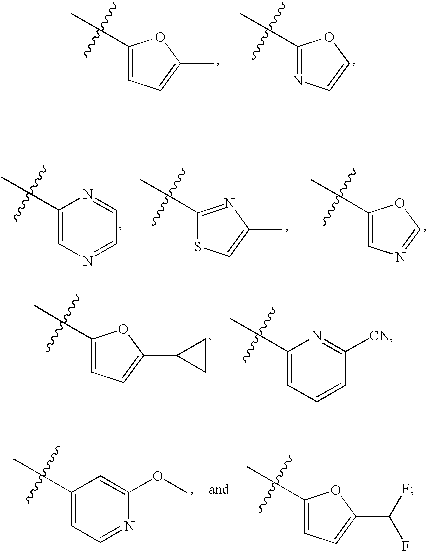
R² is selected from the group consisting of: 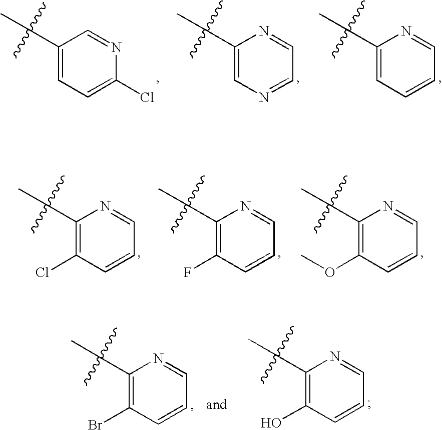
and solvates, hydrates, tautomers and pharmaceutically acceptable salts thereof.

6. A compound selected from the group consisting of: 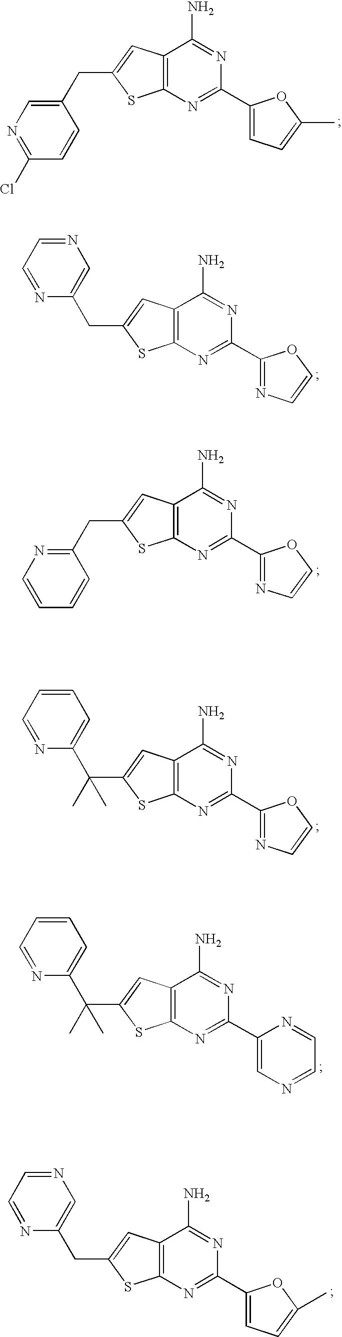 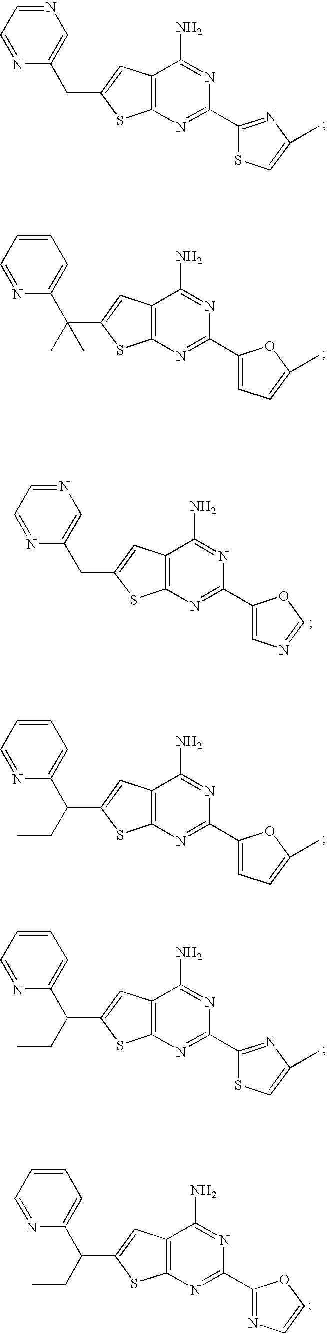 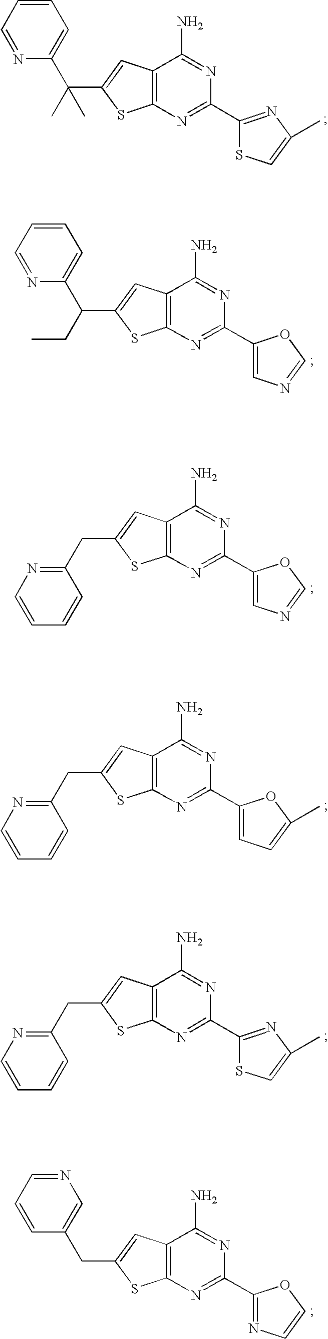 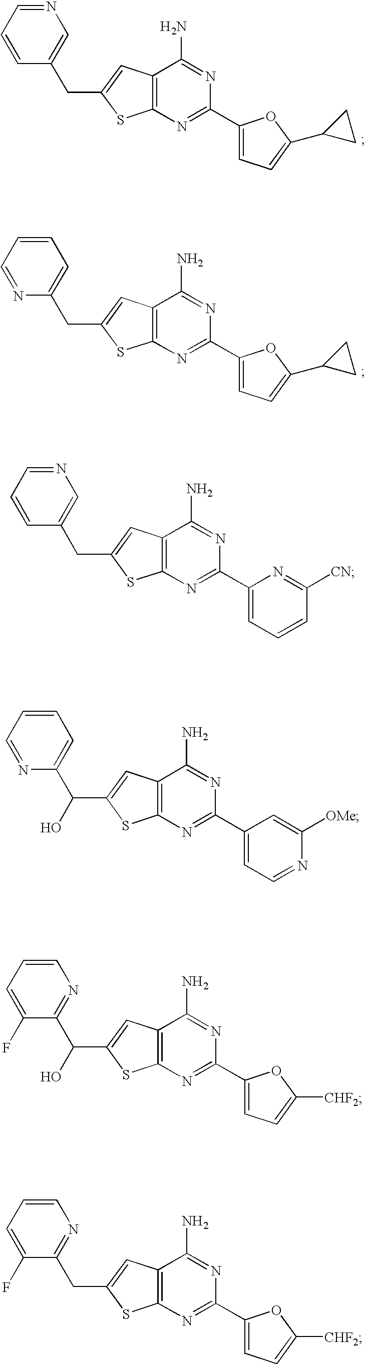 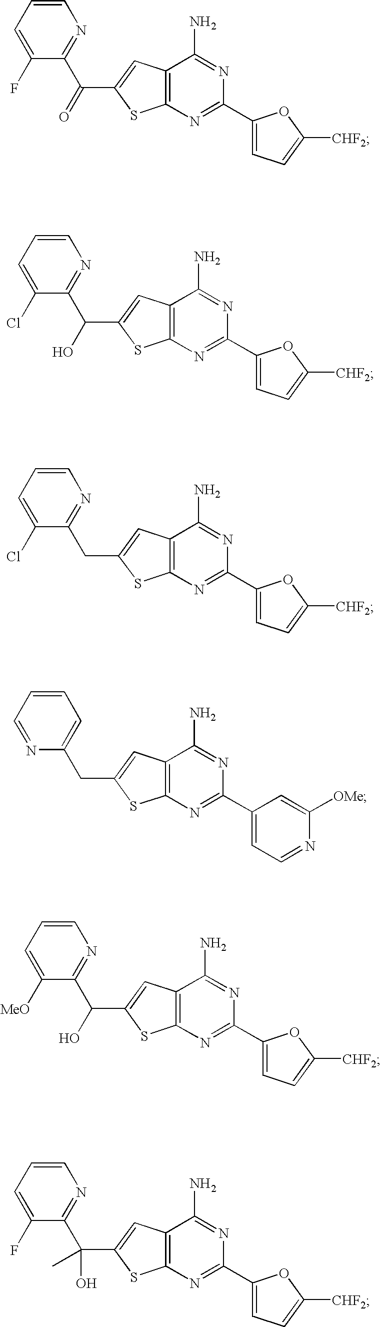 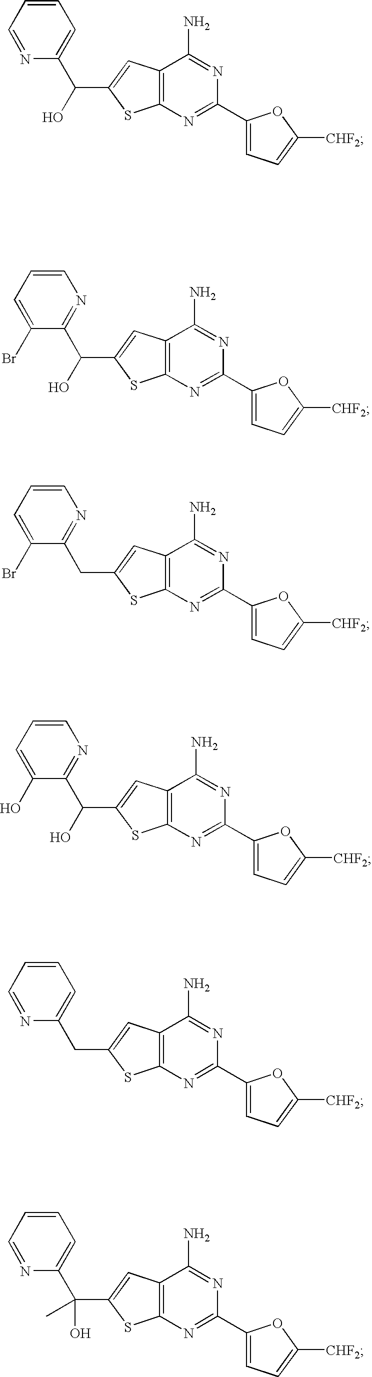 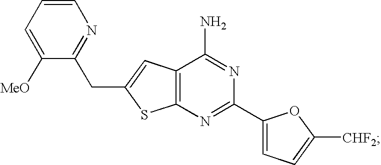
and solvates, hydrates, tautomers, and pharmaceutically acceptable salts thereof

7. A pharmaceutical composition comprising the compound of claim 1; and a pharmaceutically acceptable carrier.

8. A method of treating a subject having a disorder ameliorated by antagonizing Adenosine A2a receptors in appropriate cells in the subject, which comprises administering to the subject a therapeutically effective dose of the compound of claim 1.

9. A method of preventing a disorder ameliorated by antagonizing Adenosine A2a receptors in appropriate cells in the subject, comprising administering to the subject a prophylactically effective dose of the compound of claim 1 either preceding or subsequent to an event anticipated to cause a disorder ameliorated by antagonizing Adenosine A2a receptors in appropriate cells in the subject.

10. The method of treating a subject having a disorder ameliorated by antagonizing Adenosine A2a receptors in appropriate cells in the subject, comprising administering to the subject a therapeutically or prophylactically effective dose of the pharmaceutical composition of claim 7.

11. The method of preventing a disorder ameliorated by antagonizing Adenosine A2a receptors in appropriate cells in the subject, comprising administering to the subject a therapeutically or prophylactically effective dose of the pharmaceutical composition of claim 7.

12. The method of claim 8, wherein the disorder is a neurodegenerative disorder or a movement disorder.

13. The method of claim 8, wherein the disorder is selected from the group consisting of Parkinson's Disease, Huntington's Disease, Multiple System Atrophy, Corticobasal Degeneration, Alzheimer's Disease, and Senile Dementia.

14. The method of claim 9, wherein the disorder is a neurodegenerative disorder or a movement disorder.

15. The method of claim 9, wherein the disorder is selected from the group consisting of Parkinson's Disease, Huntington's Disease, Multiple System Atrophy, Corticobasal Degeneration, Alzheimer's Disease, and Senile Dementia.

16. The method of claim 8, wherein the disorder is Parkinson's Disease.

17. The method of claim 8, wherein the disorder is addiction.

18. The method of claim 8, wherein the disorder is Attention Deficit Hyperactivity Disorder (ADHD).

19. The method of claim 8, wherein the disorder is depression.

20. The method of claim 8, wherein the disorder is anxiety.
